(19) United States
(12) Patent Application Publication
(10) Pub. No.: US 2012/0178129 A1
(43) Pub. Date: Jul. 12, 2012

(54) GENE SYNTHESIS METHOD

(76) Inventors: Mo Huang Li, Tucson, AZ (US); Jackie Y. Yang, Singapore (SG); Chye Cheong Wai, Singapore (SG); Marcus Bode, Hamburg (DE)

(21) Appl. No.: 13/320,255

(22) PCT Filed: May 11, 2009
(86) PCT No.: PCT/SG09/00169

§ 371 Date: Mar. 23, 2012

(51) Int. Cl. C12P 19/34; C07H 21/04

(52) U.S. Cl. 435/91.2; 536/24.33

(57) ABSTRACT

The present invention relates to polymerase chain reaction (PCR)-based methods for the one-step synthesis of nucleic acid molecules, wherein the amplification primers used in said methods are designed such that they have two distinct melting temperatures in order to minimize the competition between polymerase cycling assembly (PCA) and polymerase chain reaction (PCR) amplification in the one-step nucleic acid synthesis and to maximize the emerging full-length amplification, as well as kits for use in such methods.

### FIELD OF THE INVENTION

The present invention relates to polymerase chain reaction (PCR)-based methods for the synthesis of nucleic acid molecules as well as kits for use in such methods.

### BACKGROUND OF THE INVENTION

De novo gene synthesis is a powerful molecular tool for creating and modifying genes and has broad applications in protein engineering (He, M., Stoevesandt, O., Palmer, E. A., Khan, F., Ericsson, O. and Taussig, M. J. (2008) Printing protein arrays from DNA arrays. Nat. Methods, 5, 175-177; Ramachandran, N., Raphael, J. V., Hainsworth, E., Demirkan, G., Fuentes, M. G., Rolfs, A., Hu, Y. and LaBaer, J. (2008) Next-generation high-density self-assembling functional protein arrays. Nat. Methods, 5, 535-538), development of artificial gene networks (Sprinzak, D. and Elowitz, M. B. (2005) Reconstruction of genetic circuits. Nature, 438, 443-448; Basu, S., Gerchman, Y., Collins, C. H., Arnold, F. H. and Weiss, R. (2005) A synthetic multicellular system for programmed pattern formation. Nature, 434, 1130-1134), and creation of synthetic genomes (Smith, H. O., Hutchison, C. A., III, Pfannkoch, C. and Venter, J. C. (2003) Generating a synthetic genome by whole genome assembly: ΦX174 bacteriophage from synthetic oligonucleotides. Proc. Natl. Acad. Sci. USA, 100, 15440-15445; Gibson, D. G., Benders, G. A., Andrews-Pfannkoch, C., Denisova, E. A., Baden-Tillson, H., Zaveri, J., Stockwell, T. B., Brownley, A., Thomas, D. W., Algire, M. A., Merryman, C., Young, L., Noskov, V. N., Glass, J. I., Venter, J. C., Hutchison, C. A., III and Smith, H. O. (2008) Complete chemical synthesis, assembly, and cloning of a Mycoplasma genitalium genome. Science, 319, 1215-1220; Cello, J., Paul, A. V. and Wimmer, E. (2002) Chemical synthesis of poliovirus cDNA: Generation of infectious virus in the absence of natural template. Science, 297, 1016-1018). In contrast to that, existing molecular biology techniques such as gene cloning often involve a PCR step to generate the desired gene, and thus require a DNA template. However, natural occurring template DNA is not always available for numerous reasons including lack of access to the relevant source organism, limited environmental or archaeological samples, and degradation of DNA samples or hazards associated with the natural source organism (Smith, H. O., Hutchison, C. A., III, Pfannkoch, C. and Venter, J. C. (2003) Generating a synthetic genome by whole genome assembly: ΦX174 bacteriophage from synthetic oligonucleotides. Proc. Natl. Acad. Sci. USA, 100; 15440-15445). With the ability to synthesize genes de novo in a laboratory, scientists no longer have to rely on the availability and accessibility of natural DNA.

The gene synthesis technology enables scientists to design and chemically synthesize long DNA molecules, thus allowing mutations and restriction sites to be introduced, or codon usage to be altered to match the known codon preferences of a host cell system (Hoover, D. M. and Lubkowski, J. (2002) DNAWorks: An automated method for designing oligonucleotides for PCR-based gene synthesis. Nucleic Acids Res., 30, e43; Prodromou, C. and Pearl, L. (1992) Recursive PCR: A novel technique for total gene synthesis. Protein Eng., 5, 827-829). Thus, then synthesized artificial genes facilitate the study of gene function and improve protein expression compared to using naturally occurring gene sequence as templates (Cox, J. C., Lape, J., Sayed, M. A. and Helling a, H. W. (2007) Protein fabrication automation. Protein Sci., 16, 379-390; Klammt, C., Schwarz, D., Löhr, F., Schneider, B., Dötsch, V., and Bernhard, F. (2006) Cell-free expression as an emerging technique for the large scale production of integral membrane protein. FEBS J., 273, 4141-4153).

Current gene synthesis methods include ligase chain reaction (LCR) (Smith et al., supra; Au, L. C., Yang, F. Y., Yang, W. J., Lo, S. H. and Kao, C. F. (1998) Gene synthesis by a LCR-based approach: High-level production of leptin-L54 using synthetic gene in Escherichia coli. Biochem. Biophys. Res. Commun., 248, 200-203; Bang, D. and Church, G. M. (2008) Gene synthesis by circular assembly amplification. Nat. Methods, 5, 37-39) and polymerase chain reaction (PCR) assembly (Prodromou et al., supra; Kodumal, S. J., Patel, K. G., Reid, R., Menzella, H. G., Welch, M. and Santi, D. V. (2004) Total synthesis of long DNA sequences: Synthesis of a contiguous 32-kb polyketide synthase gene cluster. Proc. Natl. Acad. Sci. USA, 101, 15573-15578), both relying on the use of overlapping oligonucleotides to construct genes. In LCR assembly, adjacent oligonucleotides with no gap between consecutive oligonucleotides are ligated together, resulting in DNA extension, whereas PCR assembly utilizes the DNA polymerase to fill up gaps in the hybridized overlapping assembly oligonucleotides. Various PCR-based methods have been reported in attempt to optimize the PCR process for long DNA sequences, and to enhance the accuracy of assembly (Gao, X., Yo, P., Keith, A., Ragan, T. J. and Harris, T. K. (2003) Thermodynamically balanced inside-out (TBIO) PCR-based gene synthesis: A novel method of primer design for high-fidelity assembly of longer gene sequences. Nucleic Acids Res., 31, e143; Xiong, A.-S., Yao, Q.-H., Peng, R.-H., Li, X., Fan, H.-Q., Cheng, Z.-M. and Li, Y. (2004) A simple, rapid, high-fidelity and cost-effective PCR-based two-step DNA synthesis method for long gene sequences. Nucleic Acids Res., 32, e98; Sandhu, G. S, Aleff, R. A. and Kline, B. C. (1992) Dual asymmetric PCR: One-step construction of synthetic genes. Biotechniques, 12, 14-16; Toung, L. and Dong, Q. (2004) Two-step total gene synthesis method. Nucleic Acids Res., 32, e59; Stemmer, W. P., Crameri, A., Ha, K. D., Brennan, T. M. and Heyneker, H. L. (1995) Single-step assembly of a gene and entire plasmid from large numbers of oligodeoxyribonucleotides. Gene, 164, 49-53; Xiong, A.-S., Yao, Q.-H., Peng, R.-H., Duan, H., Li, X., Fan, H.-Q., Cheng, Z.-M. and Li, Y. (2006) PCR-based accurate synthesis of long DNA sequences. Nat. Protoc., 1, 791-797; Wu, G., Wolf, J. B., Ibrahim, A. F., Vadasz, S., Gunasinghe, M. and Freeland, S. J. (2006) Simplified gene synthesis: A one-step approach to PCR-based gene construction. J. Biotech., 124, 496-503; Xiong, A.-S., Peng, R.-H., Zhuang, J., Gao, F., Li, Y., Cheng, Z.-M., and Yao, Q.-H. (2008) Chemical gene synthesis: strategies, software, error corrections, and applications. FEMS Microbiol. Rev., 32, 522-540). Successful gene synthesis was recently reported with an oligonucleotide concentration of 10-60 nM, an outer primer concentration of 200-800 nM, and a PCR cycle number of 20-35 (Ye, H., Huang, M. C., Li, M.-H., and Ying, J. Y. (2009) Experimental analysis of gene assembly with TopDown one-step real-time gene synthesis. Nucleic Acids Res., in press).

The existence of several distinct PCR gene synthesis methods suggests that there is lack of a standard or universal method (Wu, G., Dress, L. and Freeland, S. J. (2007) Optimal encoding rules for synthetic genes: The need for a community effort. Mol. Syst. Biol., 3, 1-5). Depending on the complexity of target genes, the synthetic genes are often constructed with a one-step or two-step overlapping process. The one-step process is preferred for short DNAs 500 bp). In the one-step protocol, the amplification primers are mixed with assembly oligonucleotides in a single PCR reaction and the assembly and amplification are conducted simultaneously. Both reactions thus compete for the fixed amount of oligonucleotides and monomers (deoxynucleotide triphosphates (dNTPs)). As the outer primers also anneal with extended oligonucleotides, intermediate products with molecular weights lower than that of the complete gene are generated. This competition between assembly and amplification is particularly critical in the synthesis of long DNA molecules (Gao, X., Yo, P., Keith, A., Ragan, T. J. and Harris, T. K. (2003) Thermodynamically balanced inside-out (TBIO) PCR-based gene synthesis: A novel method of primer design for high-fidelity assembly of longer gene sequences. Nucleic Acids Res., 31, e143; Xiong, A.-S., Yao, Q.-H., Peng, R.-H., Li, X., Fan, H.-Q., Cheng, Z.-M. and Li, Y. (2004) A simple, rapid, high-fidelity and cost-effective PCR-based two-step DNA synthesis method for long gene sequences. Nucleic Acids Res., 32, e98), and can be minimized by utilizing the two-step PCR process. In the two-step PCR protocol, amplification and assembly are performed separately. In the assembly step, a pool of short oligonucleotides is assembled into a long double-stranded DNA (dsDNA) construct (termed “template”) with the desired length using polymerase cycling assembly (PCA). The assembled template DNA is then amplified in a subsequent PCR step. In order to optimize the assembly and amplification processes different PCR conditions are applied in both steps. The two-step process is thus significantly more cost-intensive and laborious than the one-step process.

Accordingly, it is an object of the present invention to provide a method that combines the simplicity and cost-effectiveness of the one-step process with the assembly efficiency of the two-step process in the synthesis of relatively long genes.

### SUMMARY OF THE INVENTION

The present invention provides a novel approach that combines the advantages of the one-step and the two-step process, while at the same time overcoming the drawbacks of the known processes. The inventive method is based on the use of amplification primers that are designed such that they have two distinct melting temperatures in order to minimize the competition between PCA and PCR amplification in the one-step gene synthesis, and to maximize the emerging full-length amplification.

In a first aspect the present invention provides a method of synthesizing a nucleic acid molecule in a PCR-based reaction, wherein the method includes

(a) assembling a nucleic acid template by PCR comprising subjecting a PCR reaction mixture comprising a set of assembly oligonucleotides and a set of amplification primers in the presence of a nucleic acid polymerase to reaction conditions that allow hybridization of the assembly oligonucleotides to each other (annealing) and nucleic acid polymerization;

wherein the set of assembly oligonucleotides comprises at least two distinct outer assembly oligonucleotides and a multitude of distinct inner assembly oligonucleotides;

wherein each of the inner assembly oligonucleotides comprises on its 5′ end a first nucleic acid sequence complementary to a nucleic acid sequence on the 5′ end of another first inner assembly oligonucleotide and, on its 3′ end, a second nucleic acid sequence complementary to a nucleic acid sequence on the 3′ end of another second inner or one of the at least two outer assembly oligonucleotides to allow hybridization to each other under hybridization conditions;

wherein each of the outer assembly oligonucleotides comprises on its 3′ end a nucleic acid sequence complementary to a nucleic acid sequence on the 3′ end of an inner assembly oligonucleotide to allow hybridization under hybridization conditions; and

wherein each of the amplification primers comprises on its 3′ end a nucleic acid sequence that is identical to a sequence on the 5′ end of an outer assembly oligonucleotide and a nucleic acid sequence that is not identical to a nucleic acid sequence of any one of the assembly oligonucleotides and not complementary to a nucleic acid sequence of any one of the assembly oligonucleotides, wherein each melting temperature of the nucleic acid sequences of the amplification primers identical to part of the sequence of an outer assembly oligonucleotide is lower than each melting temperature of the complementary sequences of the assembly oligonucleotides, and wherein each of the melting temperatures of the complete amplification primer sequences is higher than or equal to the average melting temperatures of the complementary regions of the assembly oligonucleotides or higher than or equal to the lowest melting temperature of the complementary regions of the assembly oligonucleotides; and

(b) amplifying the assembled nucleic acid template by PCR;

wherein the reaction conditions in (a) and (b) are the same; and

wherein the reaction conditions in (a) and (b) include an annealing temperature higher than each melting temperature of the nucleic acid sequences of the amplification primers that are identical to part of the sequence of an outer assembly oligonucleotide but lower than or equal to each melting temperature of the nucleic acid sequences of the complete amplification primers.

In a second aspect, the present invention relates to a kit including a set of assembly oligonucleotides and a set of amplification primers,

wherein the set of assembly oligonucleotides comprises at least two distinct outer assembly oligonucleotides and a multitude of distinct inner assembly oligonucleotides;

wherein each of the inner assembly oligonucleotides comprises on its 5′ end a first nucleic acid sequence complementary to a nucleic acid sequence on the 5′ end of another first inner assembly oligonucleotide and, on its 3′ end, a second nucleic acid sequence complementary to a nucleic acid sequence on the 3′ end of another second inner or one of the at least two outer assembly oligonucleotides to allow hybridization to each other under hybridization conditions;

wherein each of the outer assembly oligonucleotides comprises on its 3′ end a nucleic acid sequence complementary to a nucleic acid sequence on the 3′ end of an inner assembly oligonucleotide to allow hybridization under hybridization conditions; and

wherein each of the amplification primers comprises on its 3′ end a nucleic acid sequence that is identical to a sequence on the 5′ end of an outer assembly oligonucleotide and a nucleic acid sequence that is not identical to a nucleic acid sequence of any one of the assembly oligonucleotides and not complementary to a nucleic acid sequence of any one of the assembly oligonucleotides, wherein each melting temperature of the nucleic acid sequences of the amplification primers identical to part of the sequence of an outer assembly oligonucleotide is lower than each melting temperature of the complementary sequences of the assembly oligonucleotides, and wherein each of the melting temperatures of the complete amplification primer sequences is higher than or equal to the average melting temperatures of the complementary regions of the assembly oligonucleotides or higher than or equal to the lowest melting temperature of the complementary regions of the assembly oligonucleotides.

### BRIEF DESCRIPTION OF THE DRAWINGS

The invention will be better understood with reference to the detailed description when considered in conjunction with the non-limiting examples and the accompanying drawings.

FIG. 1 shows a schematic illustration of the one-step gene synthesis method of the invention combining PCR assembly and amplification into a single stage.

FIG. 2 shows the course of a real-time PCR method according to the present invention and demonstrates that the synthesis yield is dependent on the extension time. S100A4-2 (752 bp) is synthesized with various extension time from 30 s to 120 s at an annealing temperature of 70° C. (30 s) with oligonucleotide concentration of (A,C) 10 nM and (B,D) 1 nM. (A,B) Fluorescence as a function of extension time of 30 s (⋄), 60 s (▴), 90 s (♦), and 120 s (□). (C,D) The corresponding agarose gel electrophoresis results. The synthesis from 10 nM oligonucleotides reaches the plateau within 30 cycles, while the reaction from 1 nM oligonucleotides only enters the amplification phase after 30 cycles.

FIG. 3 depicts the effect of oligonucleotide assembly concentration on the successful gene synthesis. S100A4-2 (752 bp) is synthesized with various oligonucleotide concentrations ranging from 1 nM to 40 nM. All PCR are conducted with 30-s annealing at 70° C. and 90-s extension at 72° C. (A) Fluorescence as a function of PCR cycle number for oligonucleotide concentrations of 1 nM (□), 5 nM (Δ), 10 nM (▴), 15 nM (◯), 20 nM (), and 40 nM (⋄). The change in the slopes of fluorescence increment indicates the emergence of full-length template. (B) The corresponding agarose gel electrophoresis results. The arrow indicates the undesired DNA with 2× length of full-length template, generated from non-specified full-length amplification of excess PCR.

FIG. 4 illustrates the effect of varying the annealing temperature. (A,C) S100A4-2 (752 bp) and (B,D) PKB2 (1446 bp) synthesized with various annealing temperatures ranging from 58° C. to 70° C. (30 s) and 90-s extension at 72° C. (A,B) Fluorescence as a function of PCR cycle number for annealing temperatures of 58° C. (⋄), 60° C. (Δ), 62° C. (□), 65° C. (♦), 67° C. (◯), and 70° C. (▴). (C,D) The corresponding agarose gel electrophoresis results. Higher synthesis yield is obtained with a stringent assembly annealing temperature (70° C.). The slope changes in fluorescence intensity indicate the automatic switch feature in the assembly and amplification processes.

FIG. 5 shows agarose gel electrophoresis results of conventional 1-step and ATD one-step (30-cycle) gene synthesis with dNTPs concentrations of 4 mM and 0.8 mM for (A) S100A4-1 (752 bp), (B) S100A4-2 (752 bp) and (C) PKB2 (1446 bp). All PCRs are conducted with 30-s annealing at 70° C. and 90-s extension at 72° C. The concentrations of oligonucleotides and outer primers are 10 nM and 400 nM, respectively.

FIG. 6 shows agarose gel electrophoresis results of S100A4-1 (lanes 1 and 3) and S100A4-2 (lanes 2 and 4) with oligonucleotide concentrations of 10 nM and 1 nM, and PKB2 (lane 5) with 1 nM oligonucleotides. The arrow indicates the full-length DNA. Syntheses are performed with 30 and 36 cycles, respectively, for 10 nM and 1 nM oligonucleotides, with 30-s annealing at 70° C. and 90-s extension at 72° C.

FIG. 7 illustrates the effect of hybridization reaction time. Top: Agarose gel results of (A) S100A4-1, (B) S100A4-2, and (C) PKB2 synthesized with: (1) 10-s annealing (70° C.) plus 10-s extension (72° C.), and (2) 30-s annealing (70° C.) plus 90-s extension (72° C.). Bottom: The corresponding fluorescent curves for S100A4-1 (□: 20 s, ▪: 120 s), S100A4-2 (Δ: 20 s, ▴: 120 s), and PKB2 (◯: 20 s; : 120 s). The concentrations of oligonucleotides and outer primers are 10 nM and 400 nM, respectively.

FIG. 8 shows fluorescent curves of conventional 1-step (▴, ♦) and ATD one-step gene syntheses (Δ, ⋄) with dNTPs concentration of 4 mM (♦,⋄) and 0.8 mM (▴,Δ) for (A) S100A4-1 (752 bp), (B) S100A4-2 (752 bp), and (C) PKB2 (1446 bp). All PCRs are conducted with 30-s annealing at 70° C. and 90-s extension at 72° C. The concentrations of oligonucleotides and outer primers are 10 nM and 400 nM, respectively.

FIG. 9 depicts a scheme of overlapping PCR gene synthesis.

FIG. 10 shows calculated annealing possibility distribution of (A) S100A4-1 and (B) S100A4-2 at oligonucleotide concentration of 1 nM (dash line) and 10 nM (solid line). Plotted for oligonucleotides with minimum Tₘ (black line), maximum Tₘ (grey line) and average Tₘ (blue line).

FIG. 11 depicts a plot of the melting temperature versus oligonucleotide concentration for oligonucleotide sets of S100A4-1 (dash line) and S100A4-2 (solid line). Plotted for oligonucleotides with minimum Tₘ (black line), maximum Tₘ (gray line) and average Tₘ (blue line). Both oligonucleotide sets contains more than 30 different oligonucleotides. The slopes of the average Tₘ versus the logarithmic oligonucleotide concentration were ˜1.21 and 1.28 for S100A4-1 and S100A4-2, respectively.

### DETAILED DESCRIPTION OF THE INVENTION

In PCR-based gene synthesis methods, the assembly step includes hybridizing a set of assembly oligonucleotides to each other to generate a nucleic acid template for the amplification reaction. Each of the assembly oligonucleotides contains a part of the sequence of either the sense or antisense strand of the desired nucleic acid sequence. The complete set of assembly oligonucleotides usually covers the complete gene to be synthesized in that the assembly oligonucleotides taken together contain the complete sequence information. During the assembly, assembly oligonucleotides with complementary sequences hybridize to each other (anneal) and form partially double stranded nucleic acid molecules which have an annealed double stranded segment and a single stranded segment at one or both ends of the double stranded segment. These assembled molecules comprise at least two, preferably more than two assembly oligonucleotides. The strand end at the double stranded segment, usually the 3′ end, functions as a primer and the single stranded overhang segment functions as a template for the polymerase reaction so that by action of the DNA polymerase gaps in the assembled structures are filled up. In the following PCR cycles, the generated extended DNA molecules are repeatedly dissociated and re-annealed to gradually increase DNA length until the full length template of the desired sequence is generated.

The assembled full length template DNA is then amplified by a conventional PCR amplification step. In this step, primers specific for the ends of the assembled template are used and extended to amplify the target molecule.

Such gene assembly PCR methods can be performed either as a one-step process that combines PCR assembly and PCR amplification in one reaction mixture using a single set of PCR cycles for assembly and amplification or as a two-step process that involves separate reactions and PCR cycling for the assembly and amplification reactions.

The one-step gene synthesis process allows the simple and rapid production of nucleic acid molecules, since it requires only one PCR reaction. However, as the amplification oligonucleotides (primers) and assembly oligonucleotides are present in the same reaction mixture, the assembly and amplification reactions often interfere with each other, for example in that assembled intermediate products are amplified, so that the desired product is either not generated at all or only with a very low yield.

Two-step processes provide better yield of the desired product, but such processes require two distinct PCR reactions, with intervening reagent addition and isolation steps.

In known one-step PCR-based gene synthesis methods, the assembly oligonucleotides and amplification primers are commonly designed with similar melting temperatures to allow a one-step process, that is to say assembly and amplification without the need to change the reaction conditions. Since, as noted above, assembly and amplification processes occur in parallel in such methods, the amplification primers, which are present in excess to allow sufficient amplification of the template, tend to anneal with intermediates which are not full length templates, resulting in interference with the gene assembly process as well as depletion of the outer primer and mononucleotide concentration available for amplification of the full length template once it has been assembled. This depletion may lead to a premature termination of the PCR reaction (Kong, D. S., Carr, P. A, Chen, L., Zhang, S, and Jacobson, J. M. (2007) Parallel gene synthesis in a microfluidic device. Nucleic Acids Res., 35, e61; Lee, J. Y., Lim, H.-W., Yoo, S.-I., Zhang, B.-T. & Park, T. H. (2005) Efficient initial pool generation for weighted graph problems using parallel overlap assembly. Lect. Notes Comp. Sci, 3384, 215-223). In addition, internal assembly oligonucleotides which can only be extended in the normal 5′-3′ direction may be inhibitory to the amplification of the full length gene product during the amplification PCR (Prodromou et al., supra). This competitive effect between assembly oligonucleotides and amplification primers reduces the yield of the full length gene product and results in the formation of spurious products. This competitive effect is more critical for DNA with high GC content or length (Gao, X., Yo, P., Keith, A, Ragan, T. J. and Harris, T. K. (2003) Thermodynamically balanced inside-out (TBIO) PCR-based gene synthesis: A novel method of primer design for high-fidelity assembly of longer gene sequences. Nucleic Acids Res., 31, e143; Xiong, A-S., Yao, Q.-H., Peng, R.-H., Li, X., Fan, H.-Q., Cheng, Z.-M. & Li, Y. (2004) A simple, rapid, high-fidelity and cost-effective PCR-based two-step DNA synthesis method for long gene sequences. Nucleic Acids Res., 32, e98), and is eliminated in the two-step PCR process whereby the amplification and assembly are performed separately but with the extra cost and effort of fresh PCR mixture and intervening reagent addition and isolation steps.

The present invention is based on the finding that amplification primers with two distinct melting temperatures are capable of minimizing the competition between polymerase cycling assembly (PCA) and PCR amplification in the one-step gene synthesis and can thus maximize amplification of the full-length template once it has been assembled. Utilizing amplification primers designed to have two distinct melting temperatures and assembly oligonucleotides in a PCR method that includes only one annealing temperature, wherein the first melting temperature of the primers is selected such that it minimizes premature hybridization during the template assembly and wherein the second melting temperature is selected such that it allows efficient amplification of the assembled full length template, temporally separates the processes of assembly and amplification, and thus reduces the interference between PCR assembly and amplification processes in a single reaction gene synthesis. Thus, the present invention provides a PCR-based method of single reaction gene synthesis that combines the simplicity and cost-effectiveness of known one-step processes with the efficiency of separate assembly and amplification as in known two-step processes.

Consequently, in a first aspect the present invention is directed to a method of synthesizing a nucleic acid molecule by a polymerase chain reaction (PCR), comprising:

(a) assembling a nucleic acid template by PCR comprising subjecting a PCR reaction mixture comprising a set of assembly oligonucleotides and a set of amplification primers in the presence of a nucleic acid polymerase to reaction conditions that allow hybridization of the assembly oligonucleotides to each other (annealing) and nucleic acid polymerization;

wherein the set of assembly oligonucleotides comprises at least two distinct outer assembly oligonucleotides and a multitude of distinct inner assembly oligonucleotides;

wherein each of the inner assembly oligonucleotides comprises on its 5′ end a first nucleic acid sequence complementary to a nucleic acid sequence on the 5′ end of another first inner assembly oligonucleotide and, on its 3′ end, a second nucleic acid sequence complementary to a nucleic acid sequence on the 3′ end of another second inner or one of the at least two outer assembly oligonucleotides to allow hybridization to each other under hybridization conditions;

wherein each of the outer assembly oligonucleotides comprises on its 3′ end a nucleic acid sequence complementary to a nucleic acid sequence on the 3′ end of an inner assembly oligonucleotide to allow hybridization under hybridization conditions; and

wherein each of the amplification primers comprises on its 3′ end a nucleic acid sequence that is identical to a sequence on the 5′ end of an outer assembly oligonucleotide and a nucleic acid sequence that is not identical to a nucleic acid sequence of any one of the assembly oligonucleotides and not complementary to a nucleic acid sequence of any one of the assembly oligonucleotides, wherein each melting temperature of the nucleic acid sequences of the amplification primers identical to part of the sequence of an outer assembly oligonucleotide is lower than each melting temperature of the complementary sequences of the assembly oligonucleotides, and wherein each of the melting temperatures of the complete amplification primer sequences is higher than or equal to the average melting temperatures of the complementary regions of the assembly oligonucleotides or higher than or equal to the lowest melting temperature of the complementary regions of the assembly oligonucleotides; and

(b) amplifying the assembled nucleic acid template by PCR;

wherein the reaction conditions in (a) and (b) are the same; and

wherein the reaction conditions in (a) and (b) include an annealing temperature higher than each melting temperature of the nucleic acid sequences of the amplification primers that are identical to part of the sequence of an outer assembly oligonucleotide but lower than or equal to each melting temperature of the nucleic acid sequences of the complete amplification primers.

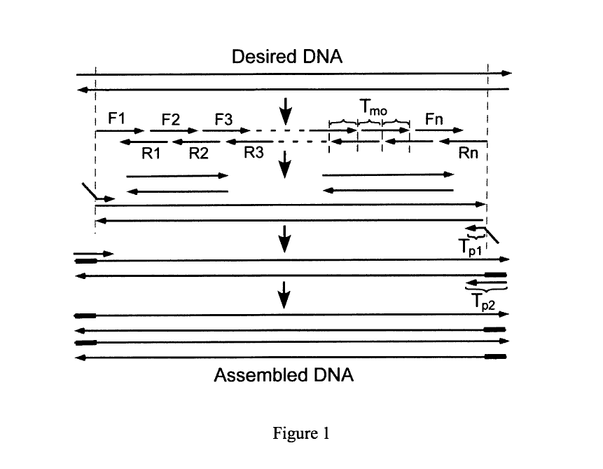

FIG. 1 is a schematic depiction of an embodiment of the present single reaction assembly and amplification PCR method.

PCR methods, conditions and reagents are well-known in the art (see, for example, U.S. Pat. Nos. 4,683,195, 4,683,202, and 4,965,188). Generally, PCR amplification is conducted in a PCR reaction mixture that includes a template nucleic acid molecule encoding the sequence that is to be amplified, primers designed such that they anneal to particular complementary target sites on the template, deoxyribonucleotide triphosphates (dNTPS), and a DNA polymerase, all combined in a suitable buffer that allows for annealing of the primers to the template and provides conditions and any cofactors or ions necessary for the DNA polymerase for primer extension.

Briefly, PCR comprises subjecting the PCR reaction mixture to thermal cycling, consisting of cycles of repeated heating and cooling of the reaction mixture for DNA melting (denaturing), annealing of the primers to the template and elongation by action of the polymerase to achieve enzymatic replication of the DNA. Generally the denaturing, annealing and elongating stages of the PCR cycle each occur at a different specific temperature and it is known in the art to conduct the PCR in a thermal cycler to achieve the required temperature for each step of the PCR cycle. Denaturing is typically performed at a temperature high enough to dissociate the DNA strands, that is to say melt any double stranded DNA (either template or amplified product formed in a previous cycle). If a heat resistant DNA polymerase, such as Taq polymerase, is used, the melting temperature can for example be as high as 95° C. The annealing step is performed at a temperature that allows the oligonucleotide primers to specifically hybridize to complementary sequences in the template DNA, and is typically chosen to allow specific hybridization while at the same time minimizing non-specific base pairing. It will be appreciated that the selection of the annealing temperature depends on the sequences of the oligonucleotides included in the PCR reaction mixture. The elongation step is performed at a temperature suitable for the particular heat-stable DNA polymerase enzyme used, to allow the DNA polymerase to enzymatically assemble a new DNA strand from mononucleotides present in the reaction mixture, by using single-stranded DNA as a template and the primers as starting points for initiation of DNA synthesis (primer extension). As the PCR progresses, the DNA generated is itself used as a template for replication, setting in motion a chain reaction in which the DNA template is exponentially amplified.

In PCR-based methods of gene synthesis that involve gene assembly, a template nucleic acid molecule is generally not provided in the PCR mixture prior to the commencement of the PCR. Rather, the template is formed during the PCR assembly stage by annealing of the pool of overlapping assembly nucleotides and extension of the overlap by the DNA polymerase to gradually synthesize longer fragments of the desired template, eventually producing a full length unbroken template after a number of PCR cycles, the number of which will depend at least in part on the length of the full length template and the number of overlapping oligonucleotides used to assemble the template.

Thus, in the present methods, it will be appreciated that the PCR reaction mixture includes the necessary components to conduct PCR (including the dNTPs, DNA polymerase and buffer), and that the template and primers are supplied in the initial reaction mixture as the set of assembly oligonucleotides and the set of amplification primers, respectively, as described below. It will also be understood that each of assembling and amplifying by PCR as described herein comprises the steps of denaturing, annealing and elongating.

As used herein, the term “oligonucleotide” refers to a single-stranded nucleic acid molecule comprising at least two nucleotides. The suitable length of an oligonucleotide for use in PCR will be known or can be readily determined by those skilled in the art. In various embodiments, the length may vary from about 10 to about 100 nucleotides and is preferably in the range of 15 to 80 nucleotides. It will be understood by a person skilled in the art that oligonucleotides can be purchased or chemically synthesized by known standard procedures.

The present PCR method involves the use of two types of oligonucleotides in the single PCR reaction mixture: assembly oligonucleotides and amplification primers.

A set of assembly oligonucleotides is any group of overlapping oligonucleotides that when annealed together produce a full-length template of a desired nucleic acid sequence or gene but having breaks or gaps along the template on alternating strands of the template, between where one oligonucleotide stops and the next oligonucleotide encoding sequence for the same strand starts. Thus, the set of assembly oligonucleotides is generally designed to cover at least the length of both strands of a double stranded DNA template, such that when all of a complete set of assembly oligonucleotides are annealed together, an annealed double stranded broken template is formed. Accordingly, the complete sequence information of the nucleic acid to be synthesized is contained within the set of assembly oligonucleotides. The set of assembly oligonucleotides utilized according to the present invention comprises at least two distinct outer assembly oligonucleotides and a multitude of distinct inner assembly oligonucleotides. As used in this context, “distinct” means that the oligonucleotides differ in their nucleotide sequence by at least one nucleotide. Each of the inner assembly oligonucleotides is complementary to either the sense or antisense strand of a portion of a desired nucleic acid sequence or gene and comprises on its 5′ end a first nucleic acid sequence complementary to a nucleic acid sequence on the 5′ end of another first inner assembly oligonucleotide and, on its 3′ end, a second nucleic acid sequence complementary to a nucleic acid sequence on the 3′ end of another second inner or one of the at least two outer assembly oligonucleotides. Each of the outer assembly oligonucleotides is complementary to either the sense or antisense strand of a portion of a desired nucleic acid sequence or gene and comprises on its 3′ end a nucleic acid sequence complementary to a nucleic acid sequence on the 3′ end of an inner assembly oligonucleotide. The outer assembly oligonucleotides may cover the sequence information of the ends of the template, e.g. comprise the sequence of the 5′ end of the sense strand of the template (first outer assembly oligonucleotide) and the sequence of the 5′ end of the antisense strand of the template, i.e. the sequence complementary to the 3′ end of the sense strand of the template (second outer assembly oligonucleotide). The complementary regions of the assembly oligonucleotides allow hybridization to each other under hybridization conditions, that is to say under annealing conditions, so as to form the double stranded full length template. As the complementary regions on the inner assembly oligonucleotides may either be adjacent or separated by a nucleotide sequence that does not hybridize to any other assembly oligonucleotide under annealing conditions, the assembled template comprises strand breaks and gaps, that are filled by the polymerase by extending the 3′ end of the hybridized assembly oligonucleotide using the single stranded part as a template.

The set of assembly oligonucleotides may be designed to produce a template having a naturally occurring sequence of a gene, or may be designed to introduce mutations or restriction sites into the final template, or to change codons to suit the codon usage of an organism in which the template DNA is ultimately to be expressed. As well, the set of assembly oligonucleotides may be designed to produce novel DNA sequences, such as DNA encoding novel fusion proteins or to insert a tag or DNA target sequence or sequence encoding a protein tag into the template DNA.

In some embodiments, the assembly oligonucleotides are each about 30 to about 100 nucleotides, about 35 to about 95, about 40 to about 90, about 45 to about 85, about 50 to about 80, about 55 to about 75, about 50 to about 70, or about 55 to about 65 nucleotides in length.

In some embodiments of the invention, the complementary regions of the assembly oligonucleotides are each about 10 to about 50, about 15 to about 45, about 20 to about 40, about 25 to about 35, or about 20 to about 30 nucleotides in length.

A set of amplification primers is a group of at least two oligonucleotides that act as primers to anneal to either strand of the full length intact template once assembled from the set of assembly oligonucleotides. The set of amplification primers facilitate PCR amplification of all or part of the full length template during the amplification stage of the present methods. In the set of amplification primers, at least one primer comprises a sequence that is complementary to a region at the 3′ end of a coding (sense) strand of the double stranded full length template and at least one amplification primer comprises a sequence that is complementary to a region at the 3′ end of a non-coding (anti-sense) strand of the double stranded full length template. As these complementary 3′ ends of the template may have to be generated during the assembly reaction by action of the polymerase, the primers may comprise sequences that are identical to the 5′ end of the outer assembly oligonucleotides. In addition to these sequence stretches that are complementary to the 3′ end of the assembled template and identical to the 5′ end of an outer assembly oligonucleotide, each of the amplification primers comprises a nucleic acid sequence that is not identical to a nucleic acid sequence of any one of the assembly oligonucleotides and not complementary to a nucleic acid sequence of any one of the assembly oligonucleotides. In this context, “not identical to a nucleic acid sequence of any one of the assembly oligonucleotides” and “not complementary to a nucleic acid sequence of any one of the assembly oligonucleotides” means that the sequence does not hybridize to any of the assembly oligonucleotides under annealing conditions. In specific embodiments of the invention, the part of the primer which hybridizes to the assembled full length template is located on the 3′ end of the primer, whereas the part of the primer that is non-complementary and non-identical to any of the assembly oligonucleotides is located on the 5′ end of the primer. In one embodiment, these two regions of the primer are directly adjacent to each other. In one specific embodiment, the sequence of the amplification primers “not identical to a nucleic acid sequence of any one of the assembly oligonucleotides” and “not complementary to a nucleic acid sequence of any one of the assembly oligonucleotides” may encode the end(s) of the gene to be synthesized, meaning that the assembly oligonucleotides do not cover the complete length of the nucleic acid to be synthesized so that the amplicons comprises the full length nucleic acid of interest.

In some embodiments, the nucleic acid sequence that is not identical to a nucleic acid sequence of any one of the assembly oligonucleotides and not complementary to a nucleic acid sequence of any one of the assembly oligonucleotides is at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length.

When hybridized to the full length template in a PCR, the amplification primers can facilitate PCR amplification of a selected portion or all of the desired nucleic acid sequence or gene.

The assembly oligonucleotides and amplification primers utilized in the inventive methods and kits are designed such that the melting temperature of each of the assembly oligonucleotides, that is to say the melting temperature of the sequence part(s) of an assembly oligonucleotide that are complementary to part(s) of another assembly oligonucleotide, is higher than each melting temperature of the sequence part of the amplification primers identical to a part of one of the outer assembly oligonucleotides. In other words, the oligonucleotides are designed such that each melting temperature of the sequence part of the amplification primers identical to a part of one of the outer assembly oligonucleotides is lower than each melting temperature of the sequence part(s) of an assembly oligonucleotide that are complementary to part(s) of another assembly oligonucleotide. The melting temperature of the part of the primer identical to the 5′ end of an outer assembly oligonucleotide is herein referred to as “first melting temperature (Tₚ₁)” of the amplification primer. The difference in melting temperatures is preferably selected such that it is sufficient to reduce the competition between PCR assembly and PCR amplification during single reaction PCR-based gene synthesis, i.e. to minimize the binding of the primers during the assembly. The melting temperature of the complete amplification primer is selected such that it can hybridize to a fully complementary sequence under annealing conditions. The melting temperature of the complete amplification primer is herein referred to as “second melting temperature (Tₚ₂)” of the amplification primer. Thus, the melting temperature of the complete amplification primer is selected such that it is equal to or even higher than the average melting temperature of the assembly oligonucleotides or, alternatively, the lowest melting temperature of the assembly oligonucleotides.

Such amplification primer design leads to very limited binding of the amplification primers during assembly, since no fully complementary targets are present at this stage of the reaction. However, once the full length template has been assembled and the amplification primers have been bound and extended, a fully complementary template strand is generated which can then be bound and amplified with high efficacy. Due to the specific design of the amplification primers, efficient amplification thus only takes place in the presence of the fully complementary template, which in turn requires a nearly completed assembly step. The specific primer design thus avoids interference of assembly and amplification and automatically initiates efficient amplification only at an advanced stage of the template assembly without the need to adapt reaction conditions. Due to this property, the inventors have termed the new method “automatic touchdown (ATD)” method.

The melting temperature of an oligonucleotide is dependent on various factors including length of the oligonucleotide and the specific nucleic acid sequence of the oligonucleotide. Therefore, the melting temperatures of the complementary region(s) of the assembly oligonucleotides may differ. Similarly, the melting temperatures of the amplification primers may differ. However, the oligonucleotides may be designed to minimize the deviation in the melting temperatures of the complementary region(s) of the assembly oligonucleotides and the deviation in the melting temperatures of the amplification primers.

The melting temperature for any given oligonucleotide can be calculated using known formulas and known programs, including commercially available software. The use of computer software to design oligonucleotides is known in the art (see, for example, US Patent Application Pub. No. 2008/0182296; Hoover, D. M. and Lubkowski, J. (2002) DNAWorks: An automated method for designing oligonucleotides for PCR-based gene synthesis. Nucleic Acids Res. 30, e43). Oligonucleotides can be designed to be optimized for increased gene expression, minimized hairpin formation and homogeneous melting temperatures (Gao et al., supra; Hoover et al., supra). For example, to design a set of assembly oligonucleotides with minimized deviation between the melting temperatures of each oligonucleotide a computer program may be used which first divides the desired nucleic acid sequence into oligonucleotides of approximately equal lengths by markers, and computes the average and deviation in melting temperatures among the overlapping regions using the nearest neighbour model with Santa Lucia's thermodynamic parameter (Santa Lucia, J., Jr. and Hicks, D. (2004) The thermodynamics of DNA structural motifs. Annu. Rev. Biophys. Biomol. Struct., 33, 415-440), corrected with salt and oligonucleotide concentrations. The oligonucleotide lengths can then be adjusted through shifting the marker positions to minimize the deviations in the melting temperatures.

In one embodiment of the invented method, the synthesized nucleic acid molecule is a double-stranded nucleic acid molecule, for example a double-stranded DNA molecule.

In one specific embodiment of the invented method the reaction conditions in (a) and (b) are identical. In a preferred embodiment of the invention, the reaction conditions during assembly and amplification are identical in that they do not include a lowering of the annealing temperature in the amplification reaction relative to that utilized in the assembly reaction.

In some embodiments of the invented methods, the difference between the melting temperatures of the complementary region(s) of the distinct assembly oligonucleotides is lower than or equal to about 10° C., lower than or equal to about 9° C., lower than or equal to about 8° C., lower than or equal to about 7° C., lower than or equal to about 6° C., lower than or equal to about 5° C., lower than or equal to about 4° C. or lower than or equal to about 3° C. In a preferred embodiment the difference is lower than 5° C. This low spread in the melting temperature of the complementary region(s) of the distinct assembly oligonucleotides allows for a very efficient assembly reaction even at assembly oligonucleotide concentrations as low as 1 nM.

In some embodiments, the average melting temperature of the complementary region(s) of the assembly oligonucleotides is in the range of about 65° C. to about 80° C. or in the range or about 70° C. to about 75° C.

An “average melting temperature” refers to the arithmetic mean of the melting temperatures of the oligonucleotides within a set of oligonucleotides, either the assembly oligonucleotides or the amplification primers, to which the average melting temperature applies. Thus, the average melting temperature of the assembly oligonucleotides is determined by averaging the melting temperatures of all the assembly oligonucleotides and the average melting temperature of the amplification primers is determined by averaging the melting temperatures of all the amplification primers. Those skilled in the art will understand that the term “melting temperature” in connection with an oligonucleotide relates to the temperature at which 50% of a population of the oligonucleotide is present in hybridized, i.e. double-stranded form, whereas the other 50% are present in dissociated, i.e. single stranded form.

As used herein, the term “about” in connection with a numerical range or concrete numerical value may relate to the given range or value ±10%, or in other some embodiments to the given range or value ±5%, or ±2%, or ±1%.

In some embodiments, the difference in the melting temperature of the complementary region(s) of each of the assembly oligonucleotides and the first melting temperature (Tₚ₁) of each of the amplification primers or, alternatively, the difference in the average melting temperature of the complementary region(s) of the assembly oligonucleotides and the average first melting temperature of the amplification primers or the first melting temperature of each of the amplification primers or, alternatively, the difference between the lowest melting temperature of the complementary region(s) of the assembly oligonucleotides and the average first melting temperature or any individual first melting temperature of the amplification primers is at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C. or at least about 25° C. In particular embodiments, the difference in the melting temperature of the complementary region(s) of each of the assembly oligonucleotides and the first melting temperature of each of the amplification primers or, alternatively, the difference in the average melting temperature of the complementary region(s) of the assembly oligonucleotides and the average first melting temperature of the amplification primers or the first melting temperature of each of the amplification primers or, alternatively, the difference between the lowest melting temperature of the complementary region(s) of the assembly oligonucleotides and the average first melting temperature or any individual first melting temperature of the amplification primers is from about 5° C. to about 20° C., or from about 5° C. to about 10° C. As noted above, “first melting temperature” refers to the melting temperature of the sequence part of an amplification primer that is identical to a part of one of the outer assembly oligonucleotides.

A person skilled in the art will recognize that the size of the difference in the melting temperatures of the complementary region(s) of each of the assembly oligonucleotides and the first melting temperatures of each of the amplification primers or, alternatively, the difference in the average melting temperature of the complementary region(s) of the assembly oligonucleotides and the average first melting temperatures of the amplification primers or the first melting temperature of each of the amplification primers or, alternatively, the difference between the lowest melting temperature of the complementary region(s) of the assembly oligonucleotides and the average first melting temperature or any individual first melting temperature of the amplification primers required for successful gene synthesis using the present method will vary depending on the annealing conditions, such as the pH and salt concentration of the PCR mixture, and the specific oligonucleotides. For example, stringent annealing conditions that reduce the likelihood of non-specific oligonucleotide annealing may permit a smaller difference in melting temperatures.

In some embodiments of the invention, the melting temperature of each of the full length amplification primers, i.e. the second melting temperature (Tₚ₂) is equal to or higher than the average melting temperature of the complementary region(s) of the assembly oligonucleotides or equal to or higher than the lowest melting temperature of the complementary region(s) of the assembly oligonucleotides. In certain embodiments, the melting temperature of each of the full length amplification primers is in the range of about 65° C. to about 80° C. or in the range or about 70° C. to about 75° C.

The PCR involves the stages of assembly and amplification, as described above. The assembly stage comprises one or more cycles of denaturing, annealing and elongating, using an annealing temperature designed to allow for assembly of the set of the assembly oligonucleotides but to reduce annealing of the amplification primers to any available complementary nucleic acid molecules that may be present. Specifically, in the assembly stage, the annealing temperature is higher than the first melting temperature (Tₚ₁) of the amplification primers to permit assembly of the assembly oligonucleotides into the full length template of the desired nucleic acid sequence, while reducing annealing of the amplification primers at this stage.

As used herein, the term “annealing temperature” refers to the temperature used during PCR to allow an oligonucleotide to form specific base pairs with a complementary strand of DNA. Typically, the annealing temperature for a particular set of oligonucleotides is chosen to be slightly below the average melting temperature, for example about 1° C., about 2° C., about 3° C. or about 5° C. below, although it may in some instances be equal to or slightly above the average melting temperature for the particular set of oligonucleotides.

In some embodiments, the annealing temperature may be chosen to be at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C. or at least about 25° C. higher than the average first melting temperature of the amplification primer set or each individual first melting temperature of the amplification primers.

In some embodiments, the annealing temperature may be chosen to be equal to or lower than the average melting temperature of the complementary region(s) of the assembly oligonucleotides.

In one embodiment, the annealing temperature may be slightly higher than the average melting temperature of the complementary region(s) of the assembly oligonucleotides. Setting the assembly annealing temperature higher than the average melting temperature of the complementary region(s) of the set of the assembly oligonucleotides may provide several advantages, including: (i) reducing potential competition between the assembly and amplification reactions, (ii) reducing the possibility of truncated oligonucleotides participating in the assembly process and the resulting errors, (iii) providing a more selective annealing condition to reduce the potential for forming secondary structures, and (iv) increasing the specialization of oligonucleotides hybridization, all of which would prevent the generation of faulty sequence, especially for genes with high GC content. It will be appreciated that the extension efficiency of some DNA polymerases is highest at 72° C. and that setting the assembly annealing temperature higher than 72° C. in the present method may reduce the assembly efficiency of the assembly oligonucleotides depending on the DNA polymerase used.

The annealing temperature is also selected such that it permits annealing of the amplification primers to a fully complementary sequence. Generally, the annealing temperature will be closer to the average second melting temperature (Tₚ₂) of the full length amplification primers than to the average melting temperature of the complementary region(s) of the assembly oligonucleotides. For example, the annealing temperature may be less than or equal to the average second melting temperature of the amplification primer set or less than or equal to each of the second melting temperatures of the amplification primers. In such embodiments, the annealing temperature may at the same time by equal to or slightly higher, that is to say about 1-10° C., preferably 2-5° C. higher than the average melting temperature of the complementary region(s) of the assembly oligonucleotides.

In the invented method, the reaction conditions do not include a lowering of the annealing temperature after the template assembly to facilitate nucleic acid amplification

As stated above, PCR conditions are generally known in the art. It will be appreciated that the reaction conditions, including for example the oligonucleotide concentration, dNTP concentration, time for each step of a cycle, number of PCR cycles, type of DNA polymerase, pH and the salt concentration of the PCR mixture, required for successful PCR will differ depending on the specific oligonucleotides and polymerase used in the reaction (see for example US Patent Application Pub. No. 2008/0182296). Thus it will be appreciated that the conditions required to achieve successful gene synthesis using the present method will vary depending on the specific assembly oligonucleotides amplification primers used and may need to be optimized for a particular reaction.

DNA polymerases that may be suitable for PCR are known in the art (Cox, J. C., Lape, J., Sayed, M. A. and Helling a, H. W. (2007) Protein fabrication automation. Protein Sci., 16, 379-390; Wu, G., Wolf, J. B., Ibrahim, A. F., Vadasz, S., Gunasinghe, M. and Freeland, S. J. (2006) Simplified gene synthesis: A one-step approach to PCR-based gene construction. J. Biotech., 124, 496-503; Mamedov, T. G., Padhye, N. V., Viljoen, H. and Subramanian, A. (2007) Rational de novo gene synthesis by rapid polymerase chain assembly (PCA) and expression of endothelial protein-C and thrombin receptor genes. J. Biotech., 131, 379-387; Arezi, B., Xing, W., Sorge, J. A. and Hogrefe, H. H. (2003) Amplification efficiency of thermostable DNA polymerase. Anal. Biochem., 321, 226-235; Cherry, J., Nieuwenhuijsen, B. W., Kaftan, E. J., Kennedy, J. D. and Chanda, P. K. (2008) A modified method for PCR-directed gene synthesis from large number of overlapping oligodeoxyribonucleotides. J. Biochem. Biophys. Methods, 70, 820-822), including for example Taq DNA polymerase, PFU DNA polymerase, hot start DNA polymerase and ProofStart™ DNA polymerase. In a particular embodiment, the KOD Hot start DNA polymerase is used in the PCR of the present method.

In some embodiments, the reaction mixture comprises the set of assembly oligonucleotides at a concentration of about 0.05 nM to about 100 nM, about 0.1 nM, about 0.2 nM, about 0.5 nM, about 1 nM, about 2 nM, about 3 nM, about 4 nM, about 5 nM, about 6 nM, about 7 nM, about 8 nM, about 9 nM, about 10 nM, about 15 nM or about 20 nM.

In some embodiments, the concentration of the set of amplification primers in the PCR mixture is from about 100 nM to about 1 μM, about 100 nM, about 200 nM, about 400 nM, about 500 nM, about 750 nM or about 1 μM.

The number of cycles required for assembly and amplification will depend at least in part on the number of oligonucleotides, the length of the template to be assembled and the uniformity of the oligonucleotides within the pool. The theoretical minimum number of cycles (x) needed in order to construct a dsDNA molecule of length (L) from uniform oligonucleotide length (n) and overlapping size (s) is given by:

2^{x}n−(2^{x}−1)s>L

In some embodiments, the number of PCR cycles for assembly of the assembly oligonucleotides is from about 5 to about 30 cycles, no less than about 5 cycles, no less than about 6 cycles, no less than about 10 cycles, no less than about 11 cycles, no less than about 15 cycles, no less than about 16 cycles, no less than about 20 cycles, no less than about 25 cycles, or no less than about 30 cycles.

In some embodiments, the number of PCR cycles for the amplification of the full length template is from about 10 to about 35 cycles, no less than about 10 cycles, no less than about 15 cycles, no less than about 20 cycles, no less than about 25 cycles, no less than about 30 cycles, or no less than about 35 cycles.

In some embodiments, the method comprises conducting from about 15 to about 50 PCR cycles.

If desired, the PCR method may begin with a “hot start”, meaning that some reagent is withheld from the reaction mixture which is then incubated at a high temperature, for example 95° C., for a short period of time before addition of the missing reagent. Hot start methods are used to reduce non-specific amplification during the initial set up stages of the PCR by restricting DNA polymerase activity until after the oligonucleotide sample has been heated to or above the oligonucleotides' melting temperature. In addition, if desired, the PCR method may end with a final extended incubation at 72° C. (see, for example, US Patent Application Pub. No. 2008/0182296).

In some embodiments of the invention, the nucleic acid molecule to be synthesized is about 500 to about 4000 nucleotides, about 1000 to about 3000 nucleotides or about 2000 nucleotides in length.

The present method may be used to synthesize desired nucleic acid molecules or genes including long and short genes as well as nucleotide molecules encoding part of a gene sequence. The nucleic acid molecules produced using the present method may be used for a variety of purposes including but not limited to the construction of recombinant DNA, optimization of codons for increased gene expression in a particular host, mutation of promoters or transcriptions terminators, and generation of DNA for cell-free or in vitro protein synthesis.

The nucleic acid molecules synthesized by the present methods may be used to express polypeptides or proteins encoded by the synthesized nucleic acid molecules. For example, the nucleic acid sequences synthesized by the present method may be used for recombinant protein expression, construction of fusion proteins and in vitro mutagenesis. Proteins have a wide range of valuable applications in a variety of fields including medicine, pharmaceuticals, research and industry. Standard methods of in vitro protein expression are known in the art. One known method of protein expression, for example, is recombinant protein expression which involves the use of expression vectors, such as plasmids or viral vectors, containing the synthesized nucleic acid sequence to achieve protein expression in an appropriate host cell.

As stated above, the optimal conditions for achieving gene synthesis differ for different oligonucleotides. Factors such as annealing temperature, concentration of oligonucleotides and number of PCR cycles can affect the success of a PCR method, and thus it may be desirable to detect and quantify the synthesized product in order to optimize conditions. Verification of gene assembly by PCR based-methods is generally done by visualizing the final PCR product using gel electrophoresis. Using this method, verification of gene assembly is delayed until the end of the PCR and the efficiency of gene synthesis after each PCR cycle cannot be determined quantitatively.

Real-time PCR (RT-PCR) is a known technique that involves the use of fluorescence to quantify DNA amplification after each PCR cycle thus permitting continuous monitoring of PCR products throughout the PCR (Wittwer, C. T., Herrmann, M. G., Moss, A A and Rasmussen, R P. (1997) Continuous fluorescence monitoring of rapid cycle DNA amplification. BioTechniques, 22, 130-138). Generally, for RT-RCR, a PCR reaction is carried out with the addition of a fluorescent marker to the PCR mixture. After each PCR cycle, the level of fluorescence in the mixture is measured to quantify the amount of double stranded DNA product produced. Fluorescent markers that are used for RT-PCR are known in the art including sequence specific RNA or DNA fluorescent probes and double stranded DNA specific dyes (Wittwer et al., supra). RT-PCR is commonly used to monitor gene amplification from template DNA, for example in disease diagnosis (Kodumal, S. J., Patel, K. G., Reid, R., Menzella, H. G., Welch, M. and Santi, D. V. (2004) Total synthesis of long DNA sequences: Synthesis of a contiguous 32-kb polypeptide synthase gene cluster. Proc. Natl. Acad. Sci. USA, 101, 15573-15578; Au, L. C., Yang, F. Y., Yang, W. J., Lo, S. H. and Kao, C. F. (1998) Gene synthesis by a LCR-based approach: High-level production of leptin-L54 using synthetic gene in Escherichia coli. Biochem. Biophys. Res. Commun., 248, 200-203).

Using RT-PCR methods during gene assembly processes allows for optimization of conditions, including the number and length of assembly cycles. Thus, the present invention also encompasses the use of real time PCR (RT-PCR) in the methods of the present invention.

Thus there is presently provided a method comprising assembling a full length template nucleic acid molecule by RT-PCR in a PCR reaction as described above, wherein a fluorescent probe is included in the reaction mixture, wherein said fluorescent probe is selected such that the fluorescent intensity detected throughout gene assembly is linearly proportional to the length and thus the quantity of full length DNA template molecules.

This method enables optimization of the conditions for PCR-based methods of gene synthesis, verification of the synthesis of the desired nucleic acid molecule or characterization of the synthesized product. Furthermore, the use of RT-PCR enables such optimization, verification and characterization to be integrated into automated methods of gene synthesis.

Thus, by monitoring fluorescent intensity throughout the RT-PCR gene assembly reaction, it is possible to determine the amount of assembled full length DNA template after each cycle and to see the effect of adjusting denaturing, annealing, elongation temperatures, the length of denaturing, annealing, elongation segments of a reaction cycle and the number of cycles performed. In this way, an optimal amount of assembled DNA template may be made.

RT-PCR may be conducted to detect and quantify the products synthesized by PCR-based gene assembly by providing fluorescent markers with particular properties and by optimizing the concentration of such markers. In RT-PCR in gene synthesis, use of a fluorescent marker that binds equally to short and long double stranded DNA molecules results in the fluorescent intensity detected throughout gene assembly being linearly proportional to the length, and thus the quantity, of the full length assembled DNA template molecules.

RT-PCR is commonly conducted using the double stranded DNA specific dye SYBR Green I. However, this dye binds preferentially to long DNA fragments (Wittwer, C. T., Reed, G. H., Gundry, C. N., Vandersteen, J. G. and Pryor, R J. (2003) High-resolution genotyping by amplicon melting analysis using LCGreen. Clin. Chem., 49, 853860; Giglio, S., Monis, P. T. and Saint, C. P. (2003) Demonstration of preferential binding of SYBR Green I to specific DNA fragments in real-time multiplex PCR Nucleic Acids Res., 31, e136) and tends to redistribute from short DNA molecules to longer DNA molecules. During the assembly step of PCR-based gene synthesis, the PCR mixture contains double stranded DNA molecules of various lengths. Thus, during thermal cycling, the SYBR Green I dye bound to shorter pieces of DNA will translocate to the longer DNA molecules as they are synthesized (Varga, A and James, D. (2006) Real-time PCR and SYBR Green I melting curve analysis for the identification of plum pox virus strains C, EA, and W: Effect of amplicon size, melt rate, and dye translocation. J. Viral. Methods, 132, 146-153), not reflecting accurate results for gene assembly methods. As such, SYBR Green I is not a suitable fluorescent dye for RT-PCR when used in combination with PCR-based methods of gene synthesis. Despite the increase in length of the synthesized DNA molecules, the fluorescent intensity detected using SYBR Green I will remain relatively unchanged throughout the PCR cycles of the assembly step.

Thus, the fluorescent markers used to conduct RT-PCR during gene assembly should have a higher affinity for double stranded DNA then single stranded DNA and should not redistribute from short DNA molecules to long DNA molecules during thermal cycling.

Particular fluorescent dyes used to conduct RT-PCR in gene assembly may include for example, LCGreen I (Wittwer, C. T., Reed, G. H., Gundry, C. N., Vandersteen, J. G. and Pryor, R J. (2003) High-resolution genotyping by amplicon melting analysis using LCGreen. Clin. Chem., 49, 853860).

Further, the amount of fluorescent marker used may be optimized to account for the large initial quantity of DNA molecules present in PCR-based methods of gene synthesis, compared to conventional PCR. The initial quantity of DNA molecules present in PCR-based gene synthesis may be larger, by greater than 6 orders of magnitude, than that in conventional PCR amplification methods. The amount of fluorescent dye used to conduct gene synthesis by RT-PCR may be increased to enable detection of synthesized DNA molecules. For example, gene synthesis may be conducted by providing a fluorescent dye, including LCGreen I, at two times the concentration normally provided in standard PCR amplification methods.

By performing PCR gene assembly methods of gene synthesis using RT-PCR, there is provided a method for optimizing gene synthesis. Continuous monitoring of PCR products throughout the assembly and amplification steps facilitates the determination of optimal conditions for gene synthesis for a particular set of oligonucleotides. For example, gene assembly PCR methods performed with RT-PCR may permit the determination of an optimal number of cycles required to complete template assembly and amplification, thus enabling the tailoring of the PCR method to reduce unnecessary additional PCR cycling that can result in the production of spurious products (Luo, R and Zhang, D. (2007) Partial strands synthesizing leads to inevitable aborting and complicated products in consecutive polymerase chain reactions (PCRs). Sci. China Ser. C Life Sci., 50, 548). In another example, the RT-PCR based methods of gene assembly may be used to determine the optimal annealing temperature for efficient assembly of the assembly oligonucleotides. In addition, RT-PCR gene assembly methods facilitate verification of gene synthesis products after each PCR cycle and thus verification need not be delayed until after the PCR is complete.

Furthermore, when gene synthesis is performed using RT-PCR, the synthesized products may be characterized by DNA melting curve analysis. DNA melting curve analysis, in combination with RT-PCR and DNA melting simulation software (Rasmussen, J. P., Saint, C. P. and Monis, P. T. (2007) Use of DNA melting simulation software for in silico diagnostic assay design: Targeting regions with complex melting curves and confirmation by real-time PCR using intercalating dyes. BMC Bioinformatics, 8, 107-118; Blake, R D., Bizzaro, J. W., Blake, J. D., Day, G R, Delcourt, S. G., Knowles, J., Marx, K A and Santa Lucia, J., Jr. (1999) Statistical mechanical simulation of polymeric DNA melting with MELTSIM. Bioinformatics, IS, 370-375), can be used to estimate the purity and quantity of PCR products. Methods of performing DNA melting curve analysis are known in the art (Wittwer, C. T., Reed, G. H., Gundry, C. N., Vandersteen, J. G. and Pryor, R J. (2003) High-resolution genotyping by amplicon melting analysis using LCGreen. Clin. Chem., 49, 853860) and generally involve detecting the level of fluorescence while slowly heating a PCR product in order to determine the melting temperature. As each double stranded DNA has its own specific melting temperature, it will be understood by one skilled in the art that successful gene synthesis using the present method would yield a product with a single, sharp melting peak, while incomplete synthesis would result in a broad melting curve. In addition, the integrated area of the melting peak in the negative derivative of the fluorescence with respect to temperature would give the quantity of the desired full-length product (Ririe, K M., Rasmussen, R P. and Wittwer, C. T. (1997) Product differentiation by analysis of DNA melting curves during the polymerase chain reaction. Anal. Biochem., 245, 154160).

RT-PCR eliminates the need for manual visualization using gel electrophoresis to verify gene synthesis and to quantify and characterize the synthesized products. Thus using RT-PCR in gene synthesis permits the use of automated methods for optimizing gene synthesis and verifying and characterizing synthesized products. The level of fluorescence indicative of complete assembly of a particular nucleic acid molecule may be pre-determined using RT-PCR. In another example, melting curve analysis, facilitated by the use of RT-PCR, can be performed by automated methods such as a computer program thus enabling automated characterization of synthesized products that can be readily integrated into systems of automated gene synthesis including for example, lab-on-a-chip methods (U.S. Provisional Application 60/963,673).

Also contemplated are kits and commercial packages that combine a set of amplification oligonucleotides and a set of amplification primers, as described above.

In one aspect, the present invention thus features a kit comprising a set of assembly oligonucleotides and a set of amplification primers, wherein the set of assembly oligonucleotides comprises at least two distinct outer assembly oligonucleotides and a multitude of distinct inner assembly oligonucleotides; wherein each of the inner assembly oligonucleotides comprises on its 5′ end a first nucleic acid sequence complementary to a nucleic acid sequence on the 5′ end of another first inner assembly oligonucleotide and, on its 3′ end, a second nucleic acid sequence complementary to a nucleic acid sequence on the 3′ end of another second inner or one of the at least two outer assembly oligonucleotides to allow hybridization to each other under hybridization conditions; wherein each of the outer assembly oligonucleotides comprises on its 3′ end a nucleic acid sequence complementary to a nucleic acid sequence on the 3′ end of an inner assembly oligonucleotide to allow hybridization under hybridization conditions; wherein each of the amplification primers comprises on its 3′ end a nucleic acid sequence that is identical to a sequence on the 5′ end of an outer assembly oligonucleotide and a nucleic acid sequence that is not identical to a nucleic acid sequence of any one of the assembly oligonucleotides and not complementary to a nucleic acid sequence of any one of the assembly oligonucleotides, wherein each melting temperature of the nucleic acid sequences of the amplification primers identical to part of the sequence of an outer assembly oligonucleotide is lower than each melting temperature of the complementary sequences of the assembly oligonucleotides, and wherein each of the melting temperatures of the complete amplification primer sequences is higher than or equal to the average melting temperatures of the assembly oligonucleotides or higher than or equal to the lowest melting temperature of the assembly oligonucleotides.

The invention is further illustrated by the following non limiting examples and the appended figures. As one of ordinary skill in the art will readily appreciate from the disclosure of the present invention, other compositions of matter, means, uses, methods, or steps, presently existing or later to be developed that perform substantially the same function or achieve substantially the same result as the corresponding exemplary embodiments described herein may likewise be utilized according to the present invention.

### EXEMPLARY EMBODIMENT OF THE INVENTION

The present invention relates to a novel method for gene synthesis that combines the simplicity and cost-effectiveness of the one-step process, with the assembly efficiency of the two-step process in the synthesis of relatively long genes. According to the invented method primers with two distinct melting temperatures are designed to minimize the competition between PCA and PCR amplification in the one-step gene synthesis, and to maximize the emerging full-length amplification. FIG. 1 shows the concept of the inventive one-step gene assembly method, which has been termed Automatic TouchDown (ATD) gene synthesis method. As mentioned above, the amplification primers are designed with two melting temperatures (first melting temperature (Tₚ₁) and second melting temperature (Tₚ₂)) where Tₚ₁ is lower than the melting temperature of assembly oligonucleotides (Tₘₒ), and Tₚ₂ is higher than or equal to the average or lowest melting temperature of the assembly oligonucleotides, such as, for example, ≧72° C. The overlapping gene synthesis is conducted in one PCR mixture with annealing temperature matched to Tₘₒ. The outer primers are subjected to an elevated annealing condition (Tₘₒ−Tₚ₁≧5° C.) during assembly, which prevents mis-pairing among primers and oligonucleotides. When the full-length template emerges, the amplification primers initially create full-length DNA with flanked tails, causing the melting temperature of amplification primer-flanked template to shift to the second melting temperature Tₚ₂ (≧72° C.). This cascade of reactions enhances the annealing possibility of the amplification primers with flanked template, and boosts the corresponding amplification of full-length template. This approach provides a unique benefit, since it automatically switches from assembly to full-length amplification as the reaction progresses. This key feature has been demonstrated by synthesizing a relatively long gene, namely human protein kinase B-2 (PKB2) (1446 bp), with single PCR from a pool of 62 assembly oligonucleotides of a concentration of as low as 1 nM. This approach presents a further improvement to the known TopDown one-step gene synthesis (Ye, H., Huang, M. C., Li, M.-H., and Ying, J. Y. (2009) Experimental analysis of gene assembly with TopDown one-step real-time gene synthesis. Nucleic Acids Res., in press).

### EXAMPLES

### 1. Experimental Procedures

### 1.1 Materials and Methods

### 1.1.1 Design of Oligonucleotides for Gene Synthesis

Gene sequences for the promoter of human calcium-binding protein A4 (S100A4, 752 bp; chr1:1503312036-1503311284) (Saleem, M., Kweon, M.-H., Johnson, J. J., Adhami, V. M., Elcheva, I. et al. (2007) S100A4 accelerates tumorigenesis and invasion of human prostate cancer through the transcriptional regulation of matrix metalloproteinase 9. Proc. Natl. Acad. Sci. USA, 103, 14825-14830) and E. coli codon-optimized human protein kinase B-2 (PKB2, 1446 bp) (Gao, X., Yo, P., Keith, A., Ragan, T. J. and Harris, T. K. (2003) Thermodynamically balanced inside-out (TBIO) PCR-based gene synthesis: A novel method of primer design for high-fidelity assembly of longer gene sequences. Nucleic Acids Res., 31, e143) were selected for synthesis via assembly PCR. Oligonucleotides were derived by a custom-developed program called TmPrime (prime.ibn.a-star.edu.sg), which would first divide the given sequence into oligonucleotides of approximately equal lengths by markers, and compute the average and deviation in melting temperatures among the overlapping regions using the nearest-neighbor model with SantaLucia's thermodynamic parameter (SantaLucia, J., Jr. and Hicks, D. (2004) The thermodynamics of DNA structural motifs. Annu. Rev. Biophys. Biomol. Struct., 33, 415-440), corrected with salt and oligonucleotide concentrations. Next, the oligonucleotide lengths were adjusted through shifting the marker positions to minimize the deviations in the overall overlapping melting temperature. Two sets of oligonucleotides (SA100A4-1 and S100A4-2) with different melting temperature uniformities (ΔTₘ: 2.3° C. and 9.1° C.) were designed to investigate the effect of melting temperature on the assembly efficiency. The oligonucleotide sets designed for the selected genes are summarized in Table 1, and their detailed information are provided in Table S1-S3.

### 1.1.2 One-Step Real-Time Gene Synthesis Method

The invented one-step process was optimized using real-time PCR conducted with Roche's LightCycler 1.5 real-time thermal cycling machine with a temperature transition of 20° C./s. Real-time gene synthesis was conducted with 20 μl of reaction mixture containing 1×PCR buffer (Novagen), 2× LCGreen I (Idaho Technology Inc.), 4 mM of MgSO₄, 1 mM each of dNTP (Stratagene), 500 μg/ml of bovine serum albumin (BSA), 1-40 nM of oligonucleotides, 400 nM of forward and reverse primers, and 1 U of KOD Hot Start (Novagen). The PCRs were conducted with: 2 min of initial denaturation at 95° C.; 30 cycles of 95° C. for 5 s, 58-70° C. for 30 s, 72° C. for 90 s; and final extension at 72° C. for 10 min. Desalted oligonucleotides were purchased from Sigma-Aldrich without additional purification. The outer primers are summarized in Table 2 with predicted melting temperatures calculated using IDT SciTools (Owczarzy, R., Tataurov, A. V., Wu, Y., Manthey, J. A., McQuisten, K. A. Almabrazi, H. G., et al., (2008) IDT SciTools: a suite for analysis and design of nucleic acid oligomers. Nucleic Acids Res. 36, W163-W169) according to the assembly buffer condition.

### 1.1.3 Gel Electrophoresis

The synthesized products were analyzed by 1.5% agarose gel (NuSieve® GTG®, Cambrex Corporation), stained with ethidium bromide (Bio-Rad Laboratories) or SYBR Green (Invitrogen), and visualized using Typhoon 9410 variable imager (Amersham Biosciences). Gel electrophoreses were performed at 100 V for 45 min with 100 bp ladder (New England) and 5 μL of DNA samples.

### 2. Results

The assembly efficiency of PCR and LCR gene synthesis relies on the effectiveness of hybridization reaction of assembly oligonucleotides at the annealing temperature. The hybridization effectiveness, expressed as the half-time constant of the hybridization reaction of a single-stranded DNA (ssDNA) in a mixture, is a function of the number of unique oligonucleotides and the oligonucleotide concentration (Wetmur, J. G. and Fresco, J. (1991) DNA probes: applications of the principles of nucleic acid hybridization. Crit. Rev. Biochem. Mol. Biol., 26, 227-259). For normal PCR amplification, this half-time constant could be as short as few seconds, dependent on the outer primer concentration. However, this constant can be significantly increased to hundreds to thousands of seconds due to the low oligonucleotide concentration (usually 10-40 nM), and the complex assembly mixture containing several tens of oligonucleotides.

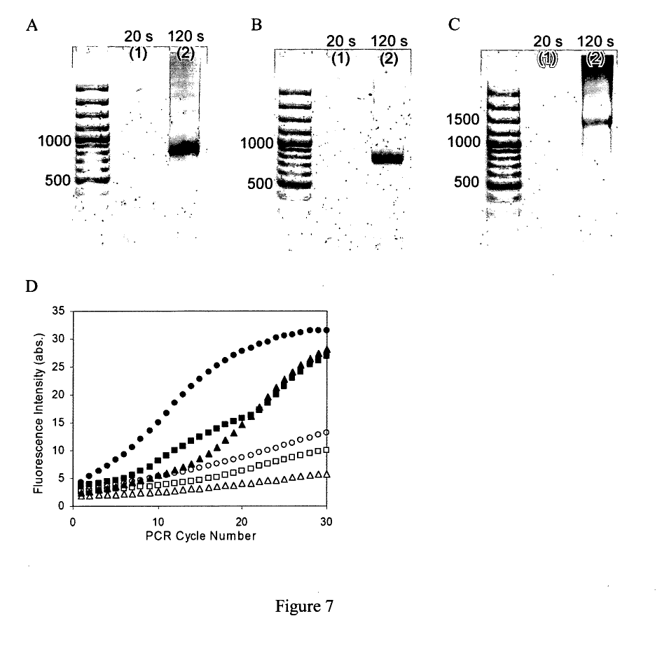

Herein, the key mechanism of reaction half-time was demonstrated by synthesizing the S100A4 (752 bp) and PKB2 (1446 bp) using a rapid thermal cycler with a temperature transition of 20° C./s. One-step gene synthesis was performed using the empirically optimized real-time gene synthesis protocol (Ye et al., supra), with either 20 s or 120 s of combined annealing (70° C.) and extension (72° C.), 2× LCGreen I, 4 mM dNTPs, and 4 mM Mg²⁺ ion. Results clearly indicated that insufficient hybridization (20-s reaction) could cause the assembly efficiency to degrade, resulting in incomplete products with DNA length of 200-300 bp (see FIG. 7).

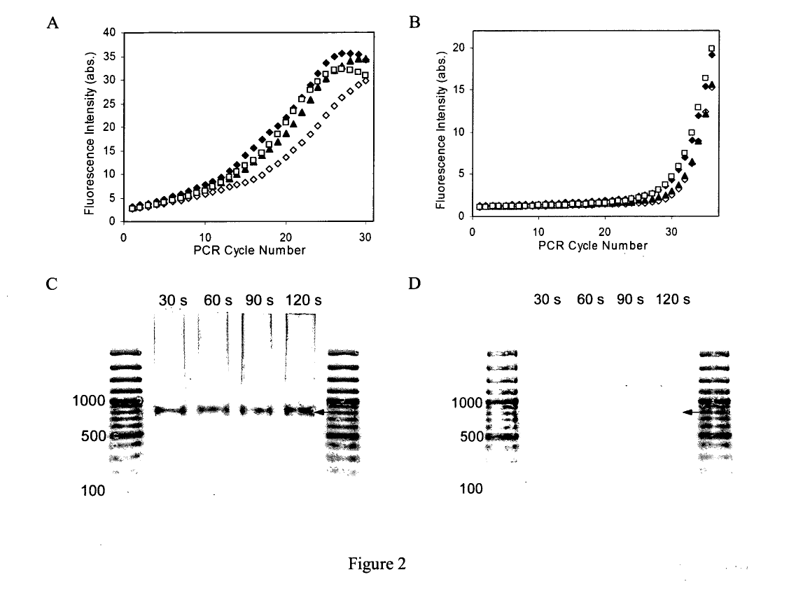

Furthermore, the effect of reaction time was investigated by varying the extension time from 30 s to 120 s for S100A4, assembled with 10 nM and 1 nM oligonucleotide, respectively. For assembly with 10 nM oligonucleotide, the reaction time was less critical. Fairly high assembly efficiency was observed where the fluorescence intensity increased as the assembly process progressed (FIGS. 2 A,C). The normal 30-s extension was sufficient to generate the full-length products, whereas prolonged extension (≧90 s) promoted the reaction so that the assembly process reached the plateau faster (in ˜25 cycles). In contrast, the assembly from 1 nM oligonucleotide has very low assembly efficiency (FIGS. 2 B,D), with a fluorescence curve like the single molecular DNA amplification (Wittwer, C. T., Herrmann, M. G., Moss, A. A. and Rasmussen, R. P. (1997) Continuous fluorescence monitoring of rapid cycle DNA amplification. BioTechniques, 22, 130-138). The gel results clearly indicated that prolonged hybridization (≧90 s) was essential for ssDNA to be effectively annealed at such a low oligonucleotide concentration.

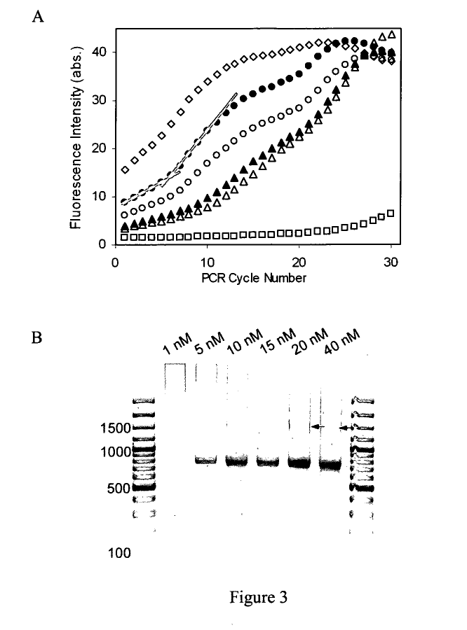

The gene synthesis took place in several phases, as revealed by the variation in slopes with the number of PCR cycles (FIG. 3). The overlapping assembly was a parallel process. Theoretically, 5 PCR cycles would be sufficient for assembling S100A4 (752 bp) from a pool of 32 oligonucleotides. Hence, relatively few PCR cycles were needed to create a full-length dsDNA. This was clearly indicated by the slope change in the fluorescent curve in the early cycles (<10 cycles). The slope became steeper as the full-length template emerged and became amplified, taking advantage of the exponential nature of PCR amplification. This phenomenon was remarkable with an oligonucleotide concentration of 5-20 nM. No obvious full-length gene product was obtained with 1 nM oligonucleotide within 30 PCR cycles, since the amplification stage was delayed due to its low assembly efficiency.

For gene synthesis with ≧20 nM of oligonucleotides, the PCR process reached the plateau within 15-20 cycles. Additional cycles would favor non-specific PCR, and lead to the build up of high molecular weight products (Gao, X., Yo, P., Keith, A., Ragan, T. J. and Harris, T. K. (2003) Thermodynamically balanced inside-out (TBIO) PCR-based gene synthesis: A novel method of primer design for high-fidelity assembly of longer gene sequences. Nucleic Acids Res., 31, e143; Xiong, A.-S., Yao, Q.-H., Peng, R.-H., Li, X., Fan, H.-Q., Cheng, Z.-M. and Li, Y. (2004) A simple, rapid, high-fidelity and cost-effective PCR-based two-step DNA synthesis method for long gene sequences. Nucleic Acids Res., 32, e98; Sandhu, G. S, Aleff, R. A. and Kline, B. C. (1992) Dual asymmetric PCR: One-step construction of synthetic genes. Biotechniques, 12, 14-16; Toung, L. and Dong, Q. (2004) Two-step total gene synthesis method. Nucleic Acids Res., 32, e59; Ye et al., supra) and the generation of spurious bands as shown in FIG. 3B (indicated by the arrow). The gel results and real-time PCR curves suggested that the optimal oligonucleotide concentration was 5-15 nM for ATD gene synthesis, which coincided with that of the conventional one-step (Wu, G., Wolf, J. B., Ibrahim, A. F., Vadasz, S., Gunasinghe, M. and Freeland, S. J. (2006) Simplified gene synthesis: A one-step approach to PCR-based gene construction. J. Biotech., 124, 496-503; Kong, D. S., Carr, P. A., Chen, L., Zhang, S, and Jacobson, J. M. (2007) Parallel gene synthesis in a microfluidic device. Nucleic Acids Res., 35, e61), TopDown one-step (Ye et al, supra) and two-step (Huang, M. C., Ye, H., Kuan, Y. K., Li, M.-H. and Ying, J. Y. (2008) Integrated two-step gene synthesis in a microfluidic device. Lab Chip, in press) processes.

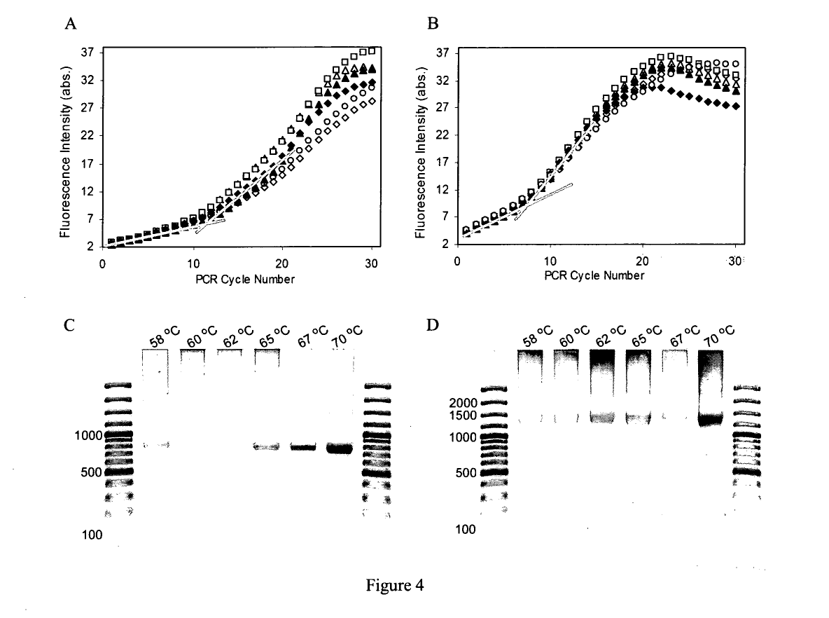

Also investigated was the effect of varying the annealing temperature from 58° C. to 70° C. (FIG. 4). The fluorescence intensity curves were indiscriminant to the annealing temperatures during the assembly phase (first 10 cycles), and began to deviate presumably only after the full-length template emerged. Interestingly, a higher yield of the desired DNA was obtained with a stringent annealing temperature (70° C.) higher than the average Tₘ of oligonucleotides (66° C.); this was consistent with the recently reported TopDown one-step process (Ye et al., supra). Performing gene synthesis at stringent annealing temperature would increase the specialization of oligonucleotide hybridization, and minimize the potential mishybridization that might occur during the gene synthesis process (see Tables S4 and S5 of the potential hybridization for S100A4 and PKB2).

The applicability of the ATD one-step process was demonstrated by synthesizing the relatively long gene, PKB2 (1446 bp), which could not be achieved by the conventional one-step gene synthesis (Gao et al., supra). Surprisingly, the PKB2 has higher assembly efficiency than that of S100A4, even although the PKB2 is ˜2× longer than S100A4. The fluorescent signal indicated the S100A4 and PKB2 syntheses reached the plateau at ˜28 and ˜22 cycles, respectively. Indeed, the ATD one-step process has fairly high assembly efficiency for oligonucleotide concentrations of 10 nM. Relatively few PCR cycles (˜10 cycles) were needed to create a full-length dsDNA, as suggested by the slope changes in fluorescent intensity in FIGS. 4 A,B. This discovery matched well with the theoretically derivation (see below), which predicted that 5 and 6 PCR cycles were sufficient for assembling S100A4 (752 bp) and PKB2 (1446 bp) from a pool of 32 and 62 oligonucleotides, respectively.

In the one-step gene synthesis process, the dNTPs could deplete and cease the PCR reaction (Owczarzy, R., Tataurov, A. V., Wu, Y., Manthey, J. A., McQuisten, K. A. Almabrazi, H. G., et al., (2008) IDT SciTools: a suite for analysis and design of nucleic acid oligomers. Nucleic Acids Res. 36, W163-W169; Lee, J. Y., Lim, H.-W., Yoo, S.-I., Zhang, B.-T. and Park, T. H. (2005) Efficient initial pool generation for weighted graph problems using parallel overlap assembly. Lect. Notes Comp. Sci., 3384, 215-223) due to the assembly-amplification interference, and the generation of a large portion of intermediate DNA products. This dNTPs depletion was critical for DNA with high GC content or length (Gao et al., supra; Xiong et al, supra). Therefore, to determine the dNTPs effects, the optimized synthesis condition determined in previous experiments were used and the gene synthesis conducted with dNTPs of 4 mM (4 mM Mg²⁺) and 0.8 mM (1.5 mM Mg²⁺) with Mg²⁺ ion (MgSO₄) concentration adjusted to compensate the dNTPs-Mg²⁺ chelation, which would affect the polymerase activity (Ely, J. J., Reeves-Daniel, A., Campbell, M. L., Kohler, S. and Stone, W. H. (1998) Influence of magnesium ion concentration and PCR amplification conditions on cross-species PCR. BioTechniques, 25, 38-40; von Ahsen, N., Wittwer, C. T. and Schütz, E. (2001) Oligonucleotide melting temperatures under PCR conditions: Nearest-neighbor corrections for Mg²⁺, deoxynucleotide triphosphate, and dimethyl sulfoxide concentrations with comparison to alternative empirical formulas. Clin. Chem., 47, 1956-1961).

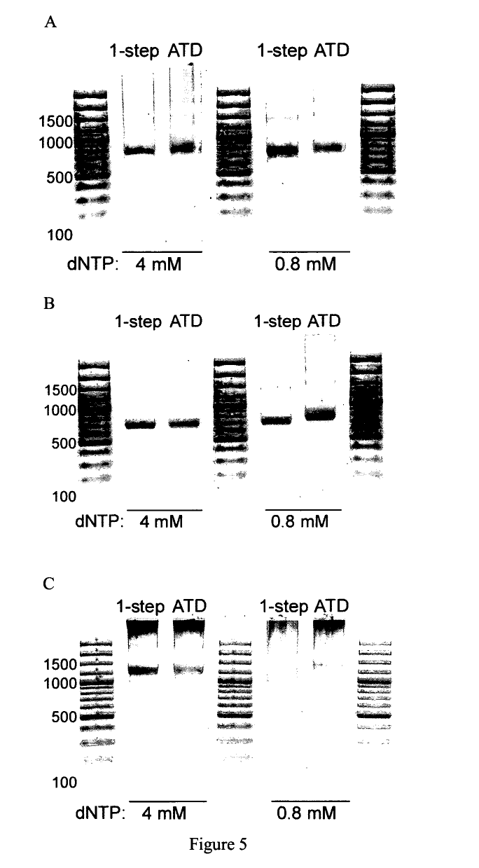

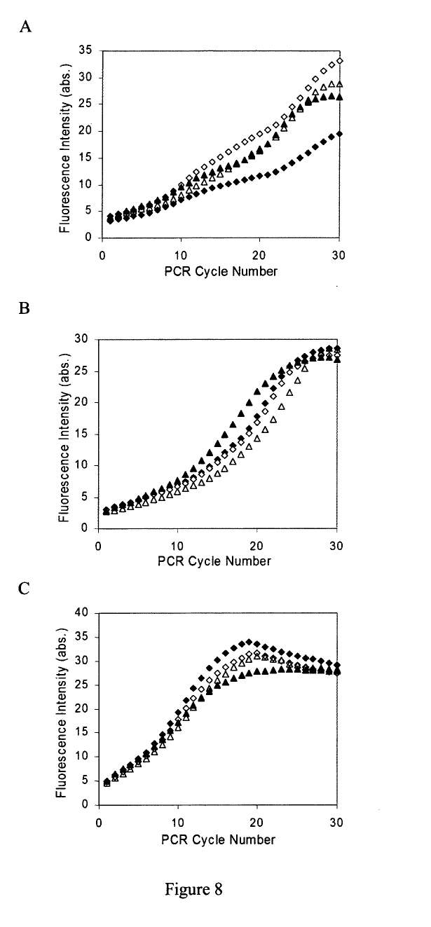

Successful gene synthesis was achieved in both of conventional one-step and ATD one-step gene synthesis of the present invention for all three genes, except the case of PKB2 with 0.8 mM dNTPs (see FIG. 5). The dNTPs concentration became more critical for relative long PKB2 where more intermediate products could be generated. The gel results and fluorescence curves (see FIG. 8) indicated that the conventional one-step process has comparable assembly efficiency with the ATD one-step for S100A4 synthesized with the optimized conditions. No obvious difference was observed for relatively short S100A4 assembled with 4 mM or 0.8 mM dNTPs in both gel results and fluorescence curves. To make the ATD a universal synthesis method for various gene lengths, 4 mM dNTPs should be used.

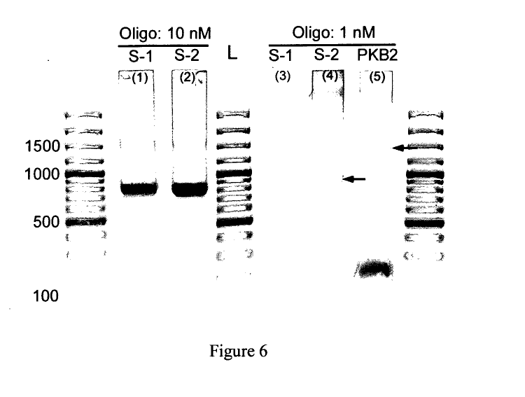

Another factor that could affect the assembly efficiency was melting temperature uniformity of assembly oligonucleotides. Two oligonucleotide sets, S100A4-1 (ΔTₘ=9.1° C.) and S100A4-2 (ΔTₘ=2.03° C.), with different Tₘ uniformity were synthesized with 10 nM and 1 nM oligonucleotide (FIG. 6). Indeed, S100A4-2 has a higher assembly efficiency than the S100A4-1. It reached the plateau within 28 cycles, whereas S100A4-1 was still in the amplification stage after 28 cycles (see FIGS. 8 A,B). However, for synthesis with ultralow oligonucleotide (1 nM), the Tₘ uniformity requirement became more essential. Only the assembly from S100A4-2 with highly uniform Tₘ was success. With this finding, successful gene synthesis was demonstrated for PKB2 (ΔTₘ=1.9° C.) with 1 nM oligonucleotide. The results suggested that the uniformity of melting temperature would be critical for ultralow oligonucleotide assembly, which has very low assembly efficiency. This is the first time that the successful gene synthesis has been achieved with an ultralow concentration of oligonucleotides of 1 nM.

### 2.1 Derivation of Minimum Cycle Number for Full-Length Assembly

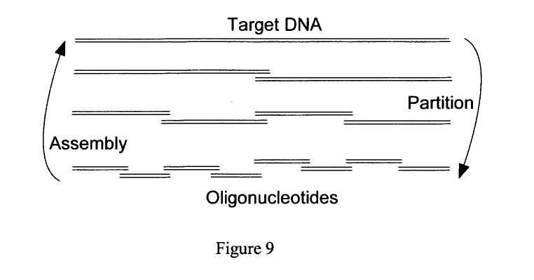

The overlapping PCR assembly is a parallel process. The lengths of overlapping oligonucleotides are extended after each PCR cycle. Careful examination of FIG. 9 reveals that the theoretical minimum number of cycles (x) in order to construct a full-length double-stranded DNA (dsDNA) molecule from a pool of n oligonucleotides can be calculated by:

x≧log₂(n)

Theoretically, 5 and 6 PCR cycles are sufficient for assembling S100A4 (752 bp) from a pool of 32 oligonucleotides, and PKB2 (1446 bp) from a pool of 62 oligonucleotides, respectively. Relatively few PCR cycles are needed to create a full-length dsDNA.

### 2.2 Derivation of Melting Temperature and Hybridization Possibility

The hybridization of two single strands of DNA is a chemical reaction that can be described using basic terms of chemistry. For short oligonucleotides, the process of DNA hybridization can be described by a two-state reaction:

S₁+S₂ [symbol] D [1]

where S₁ and S₂ represent the two single-stranded DNA, and D is a hybridized double-stranded DNA. The equilibrium constant, K, for this reaction is given by:

K=[D]/[S₁][S₂] [2]

If η is the fraction of molecule S₂ forming the duplex, the concentrations of all species can be expressed as:

[D]=η[S₂]ₒ

[S₂]=[S₂]ₒ−[D]=[S₂]ₒ(1−η)

[S₁]=[S₂]ₒ−[D]=[S₁]ₒ−η[S₂]ₒ

Therefore,

$$K = \frac{η}{( {[ {S}_{1} ]}_{o} - {η [ {S}_{2} ]}_{o} ) ( 1 - η )} \qquad [ 3 ]$$

For PCR amplification with excess out primers ([S₁]ₒ>>[S₂]ₒ), the equilibrium constant can be simplified as:

$$K = \frac{η}{{C}_{T} ( 1 - η )} , \qquad [ 4 ]$$

where C_{T} is the concentration of outer primer (S₁).

For PCR gene assembly from equal concentration of inner oligonucleotides ([S₁]ₒ=[S₂]ₒ), Eq. 3 is given by:

$$K = \frac{2 η}{{{C}_{T} ( 1 - η )}^{2}} , \qquad [ 5 ]$$

where C_{T}=[S₁]ₒ+[S₂]ₒ is the total molar strand concentration.

The annealing probability (η) can be calculated from the equilibrium constant (K) as expressed in term of Gibb's free energy change (ΔG) of this annealing reaction:

K=exp(−ΔG/RT) [6]

ΔG=ΔH−TΔS, [7]

where R is the gas constant, and ΔH and ΔS are the enthalpy and entropy changes of the annealing reaction, respectively.

The melting temperature Tₘ (K) of this reaction, defined as η=0.5, can be calculated from Eqs. 4-7.

Tₘ=ΔH/(ΔS+R×ln(C_{T}/b)) [8]

When both strands are distinct sequences with equal concentration as in the PCR assembly reaction, the value of b is 4 and K is equal to 4/C_{T} (see Eq. 5). In the case of normal PCR amplification, the value of b is 1 and K is equal to 1/C_{T}, as derived from Eq. 4.

ΔH, ΔS and ΔG of this reaction can be calculated with the following equations by using the nearest-neighbor model with SantaLucia's thermodynamic parameter (SantaLucia and Hicks, supra), corrected with salt concentrations.

ΔG[Na⁺,Mg²⁺]=ΔG[1M NaCl]−0.114×N/2×ln [Na⁺,Mg²⁺], [9]

ΔS[Na⁺,Mg²⁺]=ΔS[1M NaCl]+0.368×N/2×ln [Na⁺,Mg²⁺], [10]

[Na⁺,Mg²⁺]=[Na⁺]+4×[Mg²⁺]^{0.5} [11]

where N is the total number of phosphates in the duplex, and [Na⁺, Mg²⁺] is the concentration of sodium, potassium and magnesium cations.

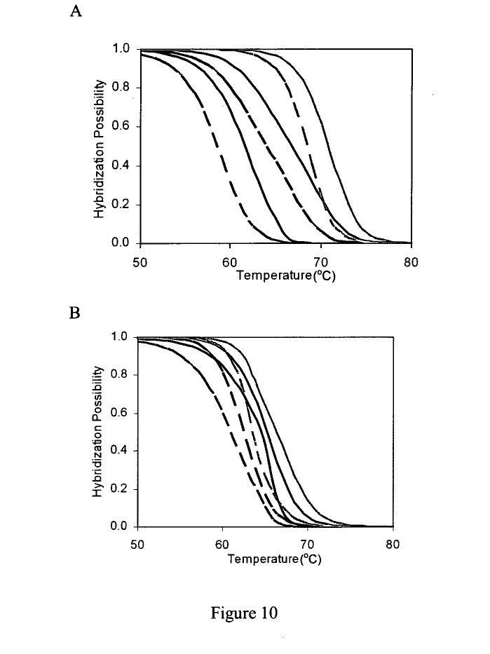

The annealing possibility curves of oligonucleotide sets of S100A4-1 and S100A4-2 were calculated from Eqs. 5 and 7 using a Matlab program with SantaLucia's thermodynamic parameter. FIG. 10 shows the relationship of annealing possibility and temperature for S100A4-1 and S100A4-2 at oligonucleotide concentration of 1 nM and 10 nM. The oligonucleotide sets were originally designed at oligonucleotide concentration of 10 nM. The average hybridization possibilities at 70° C. (annealing temperature of PCR) were 23.3% (S100A4-1) and 5.3% (S100A4-2) when oligonucleotide concentration was 10 nM, as estimated from FIG. 10. These values were reduced to 5.8% (S100A401) and 0.6% (S100A4-2), respectively, when the oligonucleotide mixture was diluted to 1 nM.

As the assembly reaction progressed, the DNA fragments became longer after each PCR cycle. The length of overlap regions and the corresponding melting temperature would increase. The hybridization curves would shift towards higher temperature. This suggested that the hybridization efficiency of DNA mixtures at the PCR annealing temperature (70° C.) might gradually improve as reaction progressed.

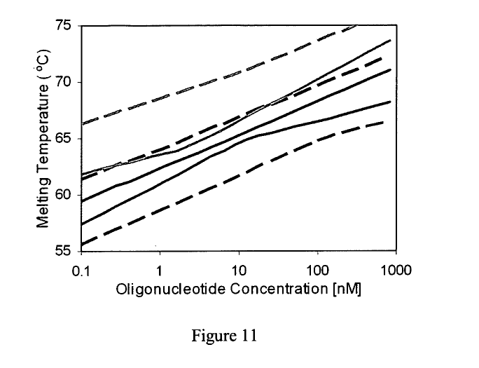

The melting temperature and oligonucleotide concentration plots for S100A-1 and S100A4-2, calculated from Eq. 8, are shown in FIG. 11. The melting temperature was approximately linearly proportional to the logarithmic oligonucleotide concentration. The melting temperatures at oligonucleotide concentration of 1 nM and 10 nM are summarized in Table S6.

For the case where R/ΔS·ln(C_{T}/b)<<1, the Tₘ can be approximated as:

$${T}_{m} = \frac{Δ H}{Δ S} ( 1 - R / Δ S \cdot ln ( {C}_{T} / b ) ) \qquad [ 12 ]$$

Based on the SantaLucia's thermodynamic parameter of the nearest-neighbor model, the average ΔH and ΔS were −8.33 kcal mol⁻¹ and −22.28 e.u., respectively. For gene assembly with an oligonucleotide concentration of 10 nM, an overlap length of 25 nt and a PCR buffer containing 50 mM NaCl and 4 mM MgCl₂, the ΔH and ΔS of the duplex calculated from Eqs. 9-11 were ˜−208.25 kcal mol⁺¹ and −583.2 e.u., respectively. By substituting these values into Eq. 12, the term of R/ΔS·ln(C_{T}/b) was found to be ˜3.4×10⁻³, and the predicted Tₘ would be give by:

Tₘ(° C.)=57.52+1.216 ln(C), [13]

where C (equal to C_{T}/2, in nM) was the oligonucleotide concentration. Based on this calculation, the melting temperature would decrease by ˜2.8° C. for every decade of reduction in oligonucleotide concentration. This value matched well with the calculated melting temperature change of S100A4-1 (2.77° C.), S100A4-2 (2.94° C.), and PKB2 (2.94° C.) as summarized in Table S6. It was noteworthy that the reduction in melting temperature has to be taken into consideration when the gene synthesis was performed with an ultralow oligonucleotide concentration of 1 nM, when the oligonucleotide sets were designed for 10 nM.

### 2.3 Kinetics of DNA Hybridization

The DNA hybridization reaction starts when that portion of two complementary ssDNA strands collides and forms a nucleation site; the rest of the sequence rapidly zippers to form a dsDNA. It has been shown that the nucleation step is the reaction limitation, and the hybridization reaction rate constant of a ssDNA in a mixture is given by [2]:

$$k = \frac{{k}_{N} \sqrt{{L}_{S}}}{N} , \qquad [ 14 ]$$

where L_{S} is the length of the shorter strand participated, k_{N} is a nucleation rate constant, and N is the complexity of the mixture, which is the number of unique oligonucleotide in the gene assembly mixture, or the primer length for standard PCR amplification.

For standard PCR amplification whereby the mixture contains only excess primers and template DNA, the hybridization reaction can be described by a pseudo-first order reaction with a half-time constant of:

$${t}_{1 / 2} = \frac{ln 2}{k {C}_{o}} \qquad [ 15 ]$$

where Cₒ is the total nucleotide concentration. Under the typical PCR amplification conditions (k_{N}≈5×10⁴/M·s) with a primer of 20 base long (L_{S}=N=20) and a primer concentration (C) of 1 μM (Cₒ=C×N), the annealing half-time is ˜3 s.

For gene assembly where the DNA is constructed from a pool of oligonucleotides with equal concentration, the hybridization reactions can be described by second-order kinetics with a half-time constant of:

$${t}_{1 / 2} = \frac{2}{k {C}_{o}} \qquad [ 16 ]$$

If we consider assembling a pool of 30 oligonucleotides (N=30) with an average length of 50 nt (L_{S}) and a concentration of 10 nM (C), the annealing half-time will be ˜339 s. In addition, the annealing half-time of outer primer (20 nt, 400 nM) will be ˜46.4 sec. For gene synthesis with an ultralow oligonucleotide concentration of 1 nM and an outer primer of 400 nM, the assembly annealing half-time dramatically increases to ˜3390 s, while the amplification half-time remains unchanged (˜46.4 s).

For overlapping PCR assembly, the average DNA length is getting longer with each PCR cycle, while the total number of strands does not change. As the reaction proceeds, various intermediate DNAs are generated from the original short oligonucleotides. Hence, the complexity (N) and <Lₛ> will increase while concentration of each DNA fragment (C) will gradually decrease. Both extendable and unextendable pairings could occur. Duplex annealed in the 3′ recessed configuration can be extended, while dsDNA annealed with 3′ ends protruded will not be extended. Unlike the exponential nature of PCR amplification, the average DNA length is most likely to increase linearly while the complexity (N) may increase more rapidly as intermediate DNAs are generated. The unextendable annealing could further complicate the assembly. Accounting for these factors, the half-time constant may increase as reaction proceeds.

The Lightcycler has an ultrafast temperature transition (20° C./s). For a typical thermocycler, the ramp rate is normally ≦4° C./s (DNA Engine PTC-200, Bio-Rad). With this thermocycler, the ramp time from 95° C. to 60° C. (annealing temperature) can take ˜8.75 s, which would be sufficient for the annealing reaction to be completed in normal PCR amplification. In addition, KOD polymerase has a very fast elongation rate (˜120 bases/s) (Takagi, M., Nishioka, M., Kakihara, H., Kitabayashi, M., Inoue, H., Kawakami, B., Oka., M. and Imanaka, T. (1997) Characterization of DNA polymerase from Pyrococcus sp. Strain KOD1 and its application to PCR. Appl. Environ. Microbiol., 63, 4505-4510). The required extension time is shorter than 10s for 1 kbp extension, which roots out the potential reaction limitation contributed by polymerase enzyme.

In summary, it is important to realize that the complexity of the assembly mixture will increase the half-life in gene assembly. The outer primer and assembly oligonucleotide have different annealing half-times that depend on their concentrations. Reducing the oligonucleotide concentration may only slightly affect its melting temperature, but it can profoundly affect the annealing kinetics. The same derivation may be applied to the ligase chain reaction (LCR) gene synthesis, which has similar underlying annealing reaction.

### 3. Discussion

The gene synthesis method disclosed herein provides a simple, rapid and low-cost approach for synthesizing long DNA (1446 bp) with only one PCR step and concentration of oligonucleotides as low as 1 nM. Experiments have demonstrated that the inventive one-step gene synthesis method was fairly efficient. The assembly process automatically switched to preferential full-length amplification as the full-length template emerged. The so-called ATD process improved the previously discussed TopDown process (Ye et al., supra) by having the PCR amplification tailored to follow the emergence of full-length DNA to avoid excess PCR.

It was found that the quality and quantity of PCR-based gene synthesis were influenced by several factors, including annealing time, annealing temperature, concentration of oligonucleotides, concentration of dNTPs monomers, and number of PCR cycles. It was also demonstrated that hybridization mechanisms of normal PCR amplification and PCR gene synthesis were different by using a rapid thermal cycler. Prolonged annealing (≧90 s) was essential for the assembly of ultralow concentration of oligonucleotides (≦1 nM), especially for long gene synthesis. The annealing duration was less critical for commonly reported gene synthesis with a DNA length of ≦500 bp and 10 nM oligonucleotides. In addition, the typical thermal cycler has a slow ramp rate of ≦4° C./s (DNA Engine PTC-200), which could contribute additional annealing time for temperature ramping from 95° C. to 60° C. With the help of the described model, insights into the optimization of gene synthesis conditions were attained. It is expected that the minimum concentration of oligonucleotides could be further reduced to 0.1 nM, which would facilitate gene synthesis using the oligonucleotides from DNA microarray (Tian, J., Gong, H., Sheng, N., Zhou, X., Gulari, E., Gao, X. and Church, G. (2004) Accurate multiplex gene synthesis from programmable DNA microchips. Nature, 2004, 432, 1050-1054; Richmond, K. E., Li, M.-H., Rodesch, M. J., Patel, M., Lowe, A. M., Kim, C., Chu, L. L., Venkataramaian, N., Flickinger, S. F., Kaysen, J., et al. (2004) Amplification and assembly of chip-eluted DNA (AACED): a method for high-throughput gene synthesis. Nucleic Acids Res., 32, 5011-5018).

The fluorescence signals indicated that an oligonucleotide concentration of 5-15 nM provided optimal assembly efficiency with a high quantity and quality of full-length products. The number of PCR cycle might have to be optimized according to sequence content and the oligonucleotide concentration to minimize the formation of abnormal products generated by excess PCR cycle (see FIG. 3). The abnormal products with incorrect DNA sequences would potentially complicate the enzymatic cleavage or the consensus shuffling error correction process (Binkowski, B. F., Richmond, K. E., Kaysen, J., Sussman, M. R. and Belshaw, P. J. (2005) Correcting errors in synthetic DNA through consensus shuffling. Nucleic Acids Res., 33, e55; Carr, P. A., Park, J. S., Lee, Y. J., Yu, T., Zhang, S, and Jacobson, J. M. (2004) Protein-mediated error correction for de novo DNA synthesis. Nucleic Acids Res., 32, e162; Fuhrmann, M., Oertel, W., Berthold, P., Hegemann, P. (2005) Removal of mismatched bases from synthetic genes by enzymatic mismatch cleavage. Nucleic Acids Res., 33, e58). Predicting the optimal PCR cycle number would be difficult, as it could rely on several factors including the complexity and length of DNA sequence, oligonucleotide concentration, annealing temperature, and Tₘ uniformity. The real-time gene synthesis with fluorescence monitoring described herein would help by providing instant feedback, terminating the process in time as it reached the plateau.

It has been found that it may be advantageous to perform the assembly with an annealing temperature slightly higher than the average melting temperature (Tₘ) of the assembly oligonucleotides. This would increase the specialization of oligonucleotides hybridization as in Touchdown PCR (Don, R. H., Cox, P. T., Wainwright, B. J., Baker, K., Mattick, J. S. (1991) ‘Touchdown’ PCR to circumvent spurious priming during gene amplification. Nucleic Acids Res., 19, 4008), and reduce the possibility of potential mis-pairing among oligonucleotides, preventing the generation of incorrect sequences. The present data also suggests that the dNTPs can be depleted for relatively long genes (kbp), and that 4 mM dNTPs should be used for universal gene synthesis. The melting temperature uniformity of assembly oligonucleotides turned out to be critical for the assembly of ultralow concentration of oligonucleotides. Therefore, it would be desirable to design the oligonucleotide sets using a bioinformatic program such as the TmPrime or DNAWorks (Hoover, D. M. and Lubkowski, J. (2002) DNAWorks: An automated method for designing oligonucleotides for PCR-based gene synthesis. Nucleic Acids Res., 30, e43).

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

### Tables

TABLE 1
Data of oligonucleotide set.

| | | Average | | Std. | | Overlap | Oligo |
|---|---|---|---|---|---|---|---|
| | Length | Tₘ | ΔTₘ | of Tₘ | # of | length | length |
| Gene | (bp) | (° C.) | (° C.) | (° C.) | oligos | (nt) | (nt) |
| S100A4-1 | 752 | 66.8 | 9.1 | 3.0 | 30 | 19-33 | 19, |
| | | | | | | | 41-66 |
| S100A4-2 | 752 | 65.2 | 2.03 | 0.48 | 32 | 18-39 | 18, |
| | | | | | | | 39-64 |
| PKB2 | 1446 | 66.2 | 1.9 | 0.59 | 62 | 16-32 | 36-57 |

TABLE 2
Summary of primers for conventional one-step, and ATD one-step gene syntheses. All PCR
assemblies are performed with an annealing temperature of 70° C.

| | Primer (5′→3′) | Tm (° C.) | Length (nt) |
|---|---|---|---|
| S100A4 | | | |
| 1-step 1 | GTTTTTCTTTCTGAATCTTTATTTTTTTAAGAGACAAG (SEQ ID NO: 1) | 62.1 | 38 |
| 1-step 2 | AAGCTTGGCCGCCG (SEQ ID NO: 2) | 58 | 14 |
| ATD 1 | AGTAGTAGTAGTAGTAGTAGTAGTAGTAGTAGTgtttttgtttctgaatctttattttttt | 69.1/55.3 | 61/28 |
| | (SEQ ID NO: 3) | | |
| ATD 2 | AGAAGAAGAAGAAGAAGAAGAAGAAGAAGAaagcttggccgccg (SEQ ID NO: 4) | 72.5/58 | 44/14 |
| PKB2 | | | |
| 1-step 1 | ATGAATGAGGTGTCTGTCATCAAAGAAGGC (SEQ ID NO: 5) | 62.9 | 30 |
| 1-step 2 | TCACTCGCGGATGCTGGCC (SEQ ID NO: 6) | 65.8 | 19 |
| ATD 1 | AGAAGAAGAAGAAGAAGAAGAAGAAGAAGAAGAatgaatgaggtgtctgtcat | 71.4/55.4 | 53/20 |
| | (SEQ ID NO: 7) | | |
| ATD 2 | AGTAGTAGTAGTAGTAGTAGTAGTAGTAGTAGTAGTtcactcgcggatgctg | 70.6/57.4 | 52/16 |
| | (SEQ ID NO: 8) | | |

TABLE S1
Semi-optimized oligonucleotides set (S100A4-1) designed for
S100A4 with oligonucleotide concentration of 10 nM.

| | | Tₘ | Overlap | Length |
|---|---|---|---|---|
| Label | Oligonucleotide sequence (5′ to 3′) | (° C.) | (bp) | (nt) |
| F1 | GTTTTTGTTTCTGAATCTTTATTTTTTTAAGAGACAAGGTCCTCTGTGTTGCTCAGGCT | 62.6 | 21 | 59 |
| | (SEQ ID NO: 9) | | | |
| R1 | TGCTCAAGCCACTGCTCTCCAGCCTGAGCAACACAGAGGAC (SEQ ID NO: 10) | 62.8 | 20 | 41 |
| F2 | GGAGAGCAGTGGCTTGAGCATAGCCAACTGCAGTCTCGAACT (SEQ ID NO: 11) | 62.0 | 22 | 42 |
| R2 | AGGAGGATCATTTGAGCCCAGGAGTTCGAGACTGCAGTTGGCTA (SEQ ID NO: 12) | 62.1 | 22 | 44 |
| F3 | CCTGGGCTCAAATGATCCTCCTGTCTCAGCTTCCTGACTAGCTGG (SEQ ID NO: 13) | 62.6 | 23 | 45 |
| R3 | GCATGGCTGTAGCCTGTAGTCCCAGCTAGTCAGGAAGCTGAGAC (SEQ ID NO: 14) | 61.1 | 21 | 44 |
| F4 | GACTACAGGCTACAGCCATGCTGCCCAGCTAATTAAAAAAAAAAATTGTTTTTC | 61.2 | 33 | 54 |
| | (SEQ ID NO: 15) | | | |
| R4 | GCAACATAGAGAGACTTCTGTCTCTATAAAAAGGAAAAACAATTTTTTTTTTTAATTAGCTGGGCA | 62.2 | 33 | 66 |
| | (SEQ ID NO: 16) | | | |
| F5 | CTTTTTATAGAGACAGAAGTCTCTCTATGTTGCCTAGGCTGGTCTTGAACTCCTGG | 62.5 | 23 | 56 |
| | (SEQ ID NO: 17) | | | |
| R5 | GAGATGGGAGGATCGCCTGAGGCCAGGAGTTCAAGACCAGCCTAG (SEQ ID NO: 18) | 64.2 | 22 | 45 |
| F6 | CCTCAGGCGATCCTCCCATCTCCCCCCTAGCTTTTGTGTCACCACATTT (SEQ ID NO: 19) | 65.8 | 27 | 49 |
| R6 | TGACAGGTGGGAGATTGCCCTGGAAATGTGGTGACACAAAAGCTAGGGGG (SEQ ID NO: 20) | 66.6 | 23 | 50 |
| F7 | CCAGGGCAATCTCCCACCTGTCACCCACCACCCCCTGCATCTCC (SEQ ID NO: 21) | 67.2 | 21 | 44 |
| R7 | GGAGTAGTCCCATGGGGACCTAGGAAAGGAGATGCAGGGGGTGGTGGG (SEQ ID NO: 22) | 66.8 | 27 | 48 |
| F8 | TTTCCTAGGTCCCCATGGGACTACTCCCTGTCCCCCATGCTCCAGGCAC (SEQ ID NO: 23) | 67.7 | 22 | 49 |
| R8 | AGGTGGAGGAAGGGGCAGCCTGTGCCTGGAGCATGGGGGACAG (SEQ ID NO: 24) | 67.9 | 21 | 43 |
| F9 | AGGCTGCCCCTTCCTCCACCTCTCTAAAACTCAGGCTGAGCTATGTACACTGGG | 67.8 | 33 | 54 |
| | (SEQ ID NO: 25) | | | |
| R9 | GGGGACTGGATGAGATGGGCACCACCCAGTGTACATAGCTCAGCCTGAGTTTTAGAG | 68.3 | 24 | 57 |
| | (SEQ ID NO: 26) | | | |
| F10 | TGGTGCCCATCTCATCCAGTCCCCTGCTAGTAACCGCTAGGGCTTACCCGTTAC | 69.2 | 30 | 54 |
| | (SEQ ID NO: 27) | | | |
| R10 | TTCCCAGGTGGGCACCCGTGGGTAACGGGTAAGCCCTAGCGGTTACTAGCA (SEQ ID NO: 28) | 69.4 | 21 | 51 |
| F11 | CCACGGGTGCCCACCTGGGAACAGGAGGCTTGGTTCCACGGCTGG (SEQ ID NO: 29) | 69.8 | 24 | 45 |
| R11 | GCCACAGCACCCTCCACCAGCCCAGCCGTGGAACCAAGCCTCCTG (SEQ ID NO: 30) | 68.5 | 21 | 45 |
| F12 | GCTGGTGGAGGGTGCTGTGGCACTTACCGCATCAGCCCACAGCAG (SEQ ID NO: 31) | 67.6 | 24 | 45 |
| R12 | GACAGGGGAGAGCGGATACTGCCTTCCTGCTGTGGGCTGATGCGGTAAGT (SEQ ID NO: 32) | 68.3 | 26 | 50 |
| F13 | GAAGGCAGTATCCGCTCTCCCCTGTCCCCTGCTATGGGCAGGGCCTG (SEQ ID NO: 33) | 67.6 | 21 | 47 |
| R13 | GCCCAGAGGTCTGACCTATTTATACCCCAGCCAGGCCCTGCCCATAGCAGGG (SEQ ID NO: 34) | 69.2 | 31 | 52 |
| F14 | GCTGGGGTATAAATAGGTCAGACCTCTGGGCCGTCCCCATTCTTCCCCTCTCTACAACC | 68.0 | 28 | 59 |
| | (SEQ ID NO: 35) | | | |
| R14 | AGATCTTGATGAAGAAGCGCTGAGGAGAGAGGGTTGTAGAGAGGGGAAGAATGGGGACG | 67.5 | 31 | 59 |
| | (SEQ ID NO: 36) | | | |
| F15 | CTCTCTCCTCAGCGCTTCTTCATCAAGATCTGGCCTCGGCGGCCAAGCTT (SEQ ID NO: 37) | 68.7 | 19 | 50 |
| R15 | AAGCTTGGCCGCCGAGGCC (SEQ ID NO: 38) | 67.7 | 19 | 19 |
| 1-Step | GTTTTTCTTTCTGAATCTTTATTTTTTAAGAGACAAG (SEQ ID NO: 1) | 59.4 | | 38 |
| F Primer | | | | |
| 1-Step | AAGCTTGGCCGCCGAGGCC (SEQ ID NO: 39) | 63.4 | | 19 |
| R Primer | | | | |
| ATD 1-Step | AGTAGTAGTAGTAGTAGTAGTAGTAGTAGTAGTgtttttgtttctgaatctttattttttt | 69.3/55.7 | 28 | 61 |
| F Primer | (SEQ ID NO: 3) | | | |
| ATD 1-Step | AGAAGAAGAAGAAGAAGAAGAAGAAGAAGAaagcttggccgccg (SEQ ID NO: 4) | 70.1/58 | 14 | 44 |
| R Primer | | | | |

TABLE S2
Optimized oligonucleotides set (S100A4-2) designed for S100A4
with oligonucleotide concentration of 10 nM.

| | | Tₘ | Overlap | Length |
|---|---|---|---|---|
| Label | Oligonucleotide sequence (5′ to 3′) | (° C.) | (bp) | (nt) |
| F1 | GMTTGITTCTGAATCTTTATTTTTTTAAGAGACAAGGTCCTCTGTGTTGCTCAGGCTGGA | 65.45 | 22 | 62 |
| | (SEQ ID NO: 40) | | | |
| R1 | GGCTATGCTCAAGCCACTGCTCTCCAGCCTGAGCAACACAGAGG (SEQ ID NO: 41) | 64.77 | 22 | 44 |
| F2 | GAGCAGTGGCTTGAGCATAGCCAACTGCAGTCTCGAACTCCTGGG (SEQ ID NO: 42) | 65.38 | 23 | 45 |
| R2 | GAAGCTGAGACAGGAGGATCATTTGAGCCCAGGAGTTCGAGACTGCAGTT (SEQ ID NO: 43) | 64.6 | 27 | 50 |
| F3 | CTCAAATGATCCTCCTGTCTCAGCTTCCTGACTAGCTGGGACTACAGGCTAC | 64.92 | 25 | 52 |
| | (SEQ ID NO: 44) | | | |
| R3 | TTTTAATTAGCTGGGCAGCATGGCTGTAGCCTGTAGTCCCAGCTAGTCAG (SEQ ID NO: 45) | 64.91 | 25 | 50 |
| F4 | AGCCATGCTGCCCAGCTAATTAAAAAAAAAAATTGMTTCCTTTTTATAGAGACAGAAGTCTC | | | |
| | (SEQ ID NO: 46) | 64.72 | 39 | 64 |
| R4 | TTCAAGACCAGCCTAGGCAACATAGAGAGACTTCTGTCTCTATAAAAAGGAAAAACAATTTTTTT | | | |
| | (SEQ ID NO: 47) | 65.06 | 26 | 65 |
| F5 | TCTATGTTGCCTAGGCTGGTCTTGAACTCCTGGCCTCAGGCGATCC (SEQ ID NO: 48) | 65.24 | 20 | 46 |
| R5 | CAAAAGCTAGGGGGGAGATGGGAGGATCGCCTGAGGCCAGGAG (SEQ ID NO: 49) | 64.78 | 23 | 43 |
| F6 | TCCCATCTCCCCCCTAGCTTTTGTGTCACCACATTTCCAGGGCAATCT (SEQ ID NO: 50) | 66.05 | 25 | 48 |
| R6 | GGTGGTGGGTGACAGGTGGGAGATTGCCCTGGAAATGTGGTGACA (SEQ ID NO: 51) | 65.59 | 20 | 45 |
| F7 | CCCACCTGTCACCCACCACCCCCTGCATCTCCTTTCCTAGGTCC (SEQ ID NO: 52) | 65.28 | 24 | 44 |
| R7 | GGGACAGGGAGTAGTCCCATGGGGACCTAGGAAAGGAGATGCAGGG (SEQ ID NO: 53) | 64.52 | 22 | 46 |
| F8 | CCATGGGACTACTCCCTGTCCCCCATGCTCCAGGCACAGGCT (SEQ ID NO: 54) | 65.73 | 20 | 42 |
| R8 | TTTTAGAGAGGTGGAGGAAGGGGCAGCCTGTGCCTGGAGCATGG (SEQ ID NO: 55) | 64.92 | 24 | 44 |
| F9 | GCCCCTTCCTCCACCTCTCTAAAACTCAGGCTGAGCTATGTACACTGGG (SEQ ID NO: 56) | 65.65 | 25 | 49 |
| R9 | GGACTGGATGAGATGGGCACCACCCAGTGTACATAGCTCAGCCTGAG (SEQ ID NO: 57) | 65.04 | 22 | 47 |
| F10 | TGGTGCCCATCTCATCCAGTCCCCTGCTAGTAACCGCTAGGGCTT (SEQ ID NO: 58) | 65.03 | 23 | 45 |
| R10 | GCACCCGTGGGTAACGGGTAAGCCCTAGCGGTTACTAGCAGG (SEQ ID NO: 59) | 65.38 | 19 | 42 |
| F11 | ACCCGTTACCCACGGGTGCCCACCTGGGAACAGGAGGCTT (SEQ ID NO: 60) | 64.99 | 21 | 40 |
| R11 | CCAGCCCAGCCGTGGAACCAAGCCTCCTGTTCCCAGGTGG (SEQ ID NO: 61) | 66.39 | 19 | 40 |
| F12 | GGTTCCACGGCTGGGCTGGTGGAGGGTGCTGTGGCACTT (SEQ ID NO: 62) | 64.93 | 20 | 39 |
| R12 | TGCTGTGGGCTGATGCGGTAAGTGCCACAGCACCCTCCA (SEQ ID NO: 63) | 65.4 | 19 | 39 |
| F13 | ACCGCATCAGCCCACAGCAGGAAGGCAGTATCCGCTCTCCC (SEQ ID NO: 64) | 65.41 | 22 | 41 |
| R13 | CCTGCCCATAGCAGGGGACAGGGGAGAGCGGATACTGCCTTCC (SEQ ID NO: 65) | 65.46 | 21 | 43 |
| F14 | CTGTCCCCTGCTATGGGCAGGGCCTGGCTGGGGTATAAATAGGTCA (SEQ ID NO: 66) | 65.28 | 25 | 46 |
| R14 | GGGGACGGCCCAGAGGTCTGACCTATTTATACCCCAGCCAGGC (SEQ ID NO: 67) | 64.6 | 18 | 43 |
| F15 | GACCTCTGGGCCGTCCCCATTCTTCCCCTCTCTACAACCCTCTCT (SEQ ID NO: 68) | 65.56 | 27 | 45 |
| R15 | CAGATCTTGATGAAGAAGCGCTGAGGAGAGAGGGTTGTAGAGAGGGGAAGAAT | 65.1 | 26 | 53 |
| | (SEQ ID NO: 69) | | | |
| F16 | CCTCAGCGCTTCTTCATCAAGATCTGGCCTCGGCGGCCAAGCTT (SEQ ID NO: 70) | 66.55 | 18 | 44 |
| R16 | AAGCTTGGCCGCCGAGGC (SEQ ID NO: 71) | 65.6 | 18 | 18 |
| 1-Step | GTTTTTCTTTCTGAATCTTTATTTTTTTAAGAGACAAG (SEQ ID NO: 1) | 59.4 | | 38 |
| F Primer | | | | |
| 1-Step | AAGCTTGGCCGCCGAGGCC (SEQ ID NO: 39) | 63.4 | | 19 |
| R Primer | | | | |
| ATD 1-Step | AGTAGTAGTAGTAGTAGTAGTAGTAGTAGTAGTgtttttgtttctgaatctttattttttt | 69.3/55.7 | 28 | 61 |
| F Primer | (SEQ ID NO: 3) | | | |
| ATD 1-Step | AGAAGAAGAAGAAGAAGAAGAAGAAGAAGAaagcttggccgccg (SEQ ID NO: 4) | 70.1/58 | 14 | 44 |
| R Primer | | | | |

TABLE S3
Oligonucleotides set designed for PKB2 with oligonucleotide concentration of 10 nM.

| | | Tₘ | Overlap | Length |
|---|---|---|---|---|
| Label | Oligonucleotide sequence (5′ to 3′) | (° C.) | (bp) | (nt) |
| F1 | ATGAATGAGGTGTCTGTCATCAAAGAAGGCTGGCTCCACAAGCGTGGTGAA | 65.71 | 21 | 51 |
| | (SEQ ID NO: 72) | | | |
| R1 | CCGTGGCCTCCAGGTCTTGATGTATTCACCACGCTTGTGGAGCCA (SEQ ID NO: 73) | 67.23 | 24 | 45 |
| F2 | TACATCAAGACCTGGAGGCCACGGTACTTCCTGCTGAAGAGCGACGG (SEQ ID NO: 74) | 65.43 | 23 | 47 |
| R2 | GCCTCTCCTTGTACCCAATGAAGGAGCCGTCGCTCTTCAGCAGGAAGTA (SEQ ID NO: 75) | 66.07 | 26 | 49 |
| F3 | CTCCTTCATTGGGTACAAGGAGAGGCCCGAGGCCCCTGATCAGACTCTA (SEQ ID NO: 76) | 65.89 | 23 | 49 |
| R3 | GCTACGGAGAAGTTGTTTAAGGGGGGTAGAGTCTGATCAGGGGCCTCGG (SEQ ID NO: 77) | 66.49 | 26 | 49 |
| F4 | CCCCCCTTAAACAACTTCTCCGTAGCAGAATGCCAGCTGATGAAGACCGAGA | 67.24 | 26 | 52 |
| | (SEQ ID NO: 78) | | | |
| R4 | AAAGGTGTTGGGTCGCGGCCTCTCGGTCTTCATCAGCTGGCATTCT (SEQ ID NO: 79) | 66.79 | 20 | 46 |
| F5 | GGCCGCGACCCAACACCTTTGTCATACGCTGCCTGCAGTGGA (SEQ ID NO: 80) | 66.05 | 22 | 42 |
| R5 | TGGAAGGTCCTCTCGATGACTGTGGTCCACTGCAGGCAGCGTATGAC (SEQ ID NO: 81) | 66.82 | 25 | 47 |
| F6 | CCACAGTCATCGAGAGGACCTTCCACGTGGATTCTCCAGACGAGAGGGA (SEQ ID NO: 82) | 66.46 | 24 | 49 |
| R6 | GGATGGCCCGCATCCACTCCTCCCTCTCGTCTGGAGAATCCACG (SEQ ID NO: 83) | 66.16 | 20 | 44 |
| F7 | GGAGTGGATGCGGGCCATCCAGATGGTCGCCAACAGCCTCAA (SEQ ID NO: 84) | 65.48 | 22 | 42 |
| R7 | GCCTGGGGCCCGCTGCTTGAGGCTGTTGGCGACCATCT (SEQ ID NO: 85) | 66.58 | 16 | 38 |
| F8 | GCAGCGGGCCCCAGGCGAGGACCCCATGGACTACAAGTGTG (SEQ ID NO: 86) | 65.82 | 25 | 41 |
| R8 | TGGAGGAGTCACTGGGGGAGCCACACTTGTAGTCCATGGGGTCCTC (SEQ ID NO: 87) | 66.23 | 21 | 46 |
| F9 | GCTCCCCCAGTGACTCCTCCACGACTGAGGAGATGGAAGTGGCG (SEQ ID NO: 88) | 66.00 | 23 | 44 |
| R9 | ACTTTAGCCCGTGCCTTGCTGACCGCCACTTCCATCTCCTCAGTCG (SEQ ID NO: 89) | 66.71 | 23 | 46 |
| F10 | GTCAGCAAGGCACGGGCTAAAGTGACCATGAATGACTTCGACTATCTCAAACTCC | 66.81 | 32 | 55 |
| | (SEQ ID NO: 90) | | | |
| R10 | ACTTTGCCAAAGGTTCCCTTGCCAAGGAGTTTGAGATAGTCGAAGTCATTCATGGTC | 67.20 | 25 | 57 |
| | (SEQ ID NO: 91) | | | |
| F11 | TTGGCAAGGGAACCTTTGGCAAAGTCATCCTGGTGCGGGAGAAGGC (SEQ ID NO: 92) | 66.25 | 21 | 46 |
| R11 | TGGCGTAGTAGCGGCCAGTGGCCTTCTCCCGCACCAGGATG (SEQ ID NO: 93) | 65.37 | 20 | 41 |
| F12 | CACTGGCCGCTACTACGCCATGAAGATCCTGCGAAAGGAAGTCATCA (SEQ ID NO: 94) | 65.69 | 27 | 47 |
| R12 | GTGTGAGCGACTTCATCCTTGGCAATGATGACTTCCTTTCGCAGGATCTTCA | 66.94 | 25 | 52 |
| | (SEQ ID NO: 95) | | | |
| F13 | TTGCCAAGGATGAAGTCGCTCACACAGTCACCGAGAGCCGGGTCC (SEQ ID NO: 96) | 66.60 | 20 | 45 |
| R13 | ACGGGTGCCTGGTGTTCTGGAGGACCCGGCTCTCGGTGACT (SEQ ID NO: 97) | 66.96 | 21 | 41 |
| F14 | TCCAGAACACCAGGCACCCGTTCCTCACTGCGCTGAAGTATGCC (SEQ ID NO: 98) | 66.00 | 23 | 44 |
| R14 | AGGCGGTCGTGGGTCTGGAAGGCATACTTCAGCGCAGTGAGGA (SEQ ID NO: 99) | 66.54 | 20 | 43 |
| F15 | TTCCAGACCCACGACCGCCTGTGCTTTGTGATGGAGTATGCCAACG (SEQ ID NO: 100) | 66.17 | 26 | 46 |
| R15 | CAGGTGGAAGAACAGCTCACCCCCGTTGGCATACTCCATCACAAAGCAC | 66.20 | 23 | 49 |
| | (SEQ ID NO: 101) | | | |
| F16 | GGGGTGAGCTGTTCTTCCACCTGTCCCGGGAGCGTGTCTTCACA (SEQ ID NO: 102) | 66.66 | 21 | 44 |
| R16 | AAAACCGGGCCCGCTCCTCTGTGAAGACACGCTCCCGGGA (SEQ ID NO: 103) | 65.79 | 19 | 40 |
| F17 | GAGGAGCGGGCCCGGITTTATGGIGCAGAGATTGTCTCGGCTC (SEQ ID NO: 104) | 65.95 | 24 | 43 |
| R17 | GTCCCGCGAGTGCAAGTACTCAAGAGCCGAGACAATCTCTGCACCAT (SEQ ID NO: 105) | 66.13 | 23 | 47 |
| F18 | TTGAGTACTTGCACTCGCGGGACGTGGTATACCGCGACATCAAGCTGG | 66.85 | 25 | 48 |
| | (SEQ ID NO: 106) | | | |
| R18 | GCCATCTTTGTCCAGCATGAGGTTTTCCAGCTTGATGTCGCGGTATACCAC | 65.72 | 26 | 51 |
| | (SEQ ID NO: 107) | | | |
| F19 | AAAACCTCATGCTGGACAAAGATGGCCACATCAAGATCACTGACTTTGGCCTCT | 66.49 | 28 | 54 |
| | (SEQ ID NO: 108) | | | |
| R19 | CCCGTCACTGATGCCCTCTTTGCAGAGGCCAAAGTCAGTGATCTTGATGTG | 67.04 | 23 | 51 |
| | (SEQ ID NO: 109) | | | |
| F20 | GCAAAGAGGGCATCAGTGACGGGGCCACCATGAAAACCTTCTGTGGG (SEQ ID NO: 110) | 65.58 | 24 | 47 |
| R20 | GCGCCAGGTACTCCGGGGTCCCACAGAAGGTTTTCATGGTGGC (SEQ ID NO: 111) | 67.11 | 19 | 43 |
| F21 | ACCCCGGAGTACCTGGCGCCTGAGGTGCTGGAGGACAATGACT (SEQ ID NO: 112) | 65.37 | 24 | 43 |
| R21 | AGTCCACGGCCCGGCCATAGTCATTGTCCTCCAGCACCTCAG (SEQ ID NO: 113) | 66.98 | 18 | 42 |
| F22 | ATGGCCGGGCCGTGGACTGGTGGGGGCTGGGTGTGG (SEQ ID NO: 114) | 65.54 | 18 | 36 |
| R22 | GGCCGCACATCATCTCGTACATGACCACACCCAGCCCCCACC (SEQ ID NO: 115) | 66.23 | 24 | 42 |
| F23 | TCATGTACGAGATGATGTGCGGCCGCCTGCCCTTCTACAACCAGGAC (SEQ ID NO: 116) | 66.26 | 23 | 47 |
| R23 | AGCTCGAAGAGGCGCTCGTGGTCCTGGTTGTAGAAGGGCAGGC (SEQ ID NO: 117) | 65.65 | 20 | 43 |
| F24 | CACGAGCGCCTCTTCGAGCTCATCCTCATGGAAGAGATCCGCTTCC (SEQ ID NO: 118) | 66.17 | 26 | 46 |
| R24 | GGGGCTGAGCGTGCGCGGGAAGCGGATCTCTTCCATGAGGATG (SEQ ID NO: 119) | 67.28 | 17 | 43 |
| F25 | CGCGCACGCTCAGCCCCGAGGCCAAGTCCCTGCTTGCT (SEQ ID NO: 120) | 65.88 | 21 | 38 |
| R25 | TTGGGGTCCTTCTTAAGCAGCCCAGCAAGCAGGGACTTGGCCTC (SEQ ID NO: 121) | 65.75 | 23 | 44 |
| F26 | GGGCTGCTTAAGAAGGACCCCAAGCAGAGGCTTGGTGGGGGG (SEQ ID NO: 122) | 65.83 | 19 | 42 |
| R26 | ACCTCCTTGGCATCGCTGGGCCCCCCACCAAGCCTCTGC (SEQ ID NO: 123) | 65.45 | 20 | 39 |
| F27 | CCCAGCGATGCCAAGGAGGTCATGGAGCACAGGTTCTTCCTCAGC (SEQ ID NO: 124) | 66.80 | 25 | 45 |
| R27 | GGACCACGTCCTGCCAGTTGATGCTGAGGAAGAACCTGTGCTCCATG (SEQ ID NO: 125) | 65.76 | 22 | 47 |
| F28 | ATCAACTGGCAGGACGTGGTCCAGAAGAAGCTCCTGCCACCCTTCA (SEQ ID NO: 126) | 66.94 | 24 | 46 |
| R28 | GACCTCGGACGTGACCTGAGGTTTGAAGGGTGGCAGGAGCTTCTTCT (SEQ ID NO: 127) | 66.97 | 23 | 47 |
| F29 | AACCTCAGGTCACGTCCGAGGTCGACACAAGGTACTTCGATGATGAATTTACCG | 65.87 | 31 | 54 |
| | (SEQ ID NO: 128) | | | |
| R29 | GGGGTGTGATTGTGATGGACTGGGCGGTAAATTCATCATCGAAGTACCTTGTGTC | 66.45 | 24 | 55 |
| | (SEQ ID NO: 129) | | | |
| F30 | CCCAGTCCATCACAATCACACCCCCTGACCGCTATGACAGCCTGGG (SEQ ID NO: 130) | 65.88 | 22 | 46 |
| R30 | TCCGCTGGTCCAGCTCCAGTAAGCCCAGGCTGTCATAGCGGTCAG (SEQ ID NO: 131) | 67.08 | 23 | 45 |
| F31 | CTTACTGGAGCTGGACCAGCGGACCCACTTCCCCCAGTTCTCCTACTC (SEQ ID NO: 132) | 66.90 | 25 | 48 |
| R31 | TCACTCGCGGATGCTGGCCGAGTAGGAGAACTGGGGGAAGTGGG (SEQ ID NO: 133) | 65.80 | 19 | 44 |
| F Primer | ATGAATGAGGTGTCTGTCATCAAAGAAGGC (SEQ ID NO: 5) | 66.97 | | 30 |
| R Primer | TCACTCGCGGATGCTGGCC (SEQ ID NO: 6) | 65.80 | | 19 |
| ATD 1-Step | AGAAGAAGAAGAAGAAGAAGAAGAAGAAGAAGAatgaatgaggtgtctgtcat | 72.7/57.2 | 20 | 53 |
| F Primer | (SEQ ID NO: 7) | | | |
| ATD 1-Step | AGTAGTAGTAGTAGTAGTAGTAGTAGTAGTAGTAGTtcactcgcggatgctg | 71.7/59 | 16 | 52 |
| R Primer | (SEQ ID NO: 8) | | | |

TABLE S4
Partial list of potential mishybridizations for SA100A4
gene synthesis predicted by TmPrime
gene synthesis software (http://prime.ibn.a-star.edu.sg).
The oligonucleotides are alternately displayed
in upper and lower case for ease of finding the oligonucleotide boundaries.
Both the forward and
reverse mishybridizations are reported, which have the same
number of matched bases, but may
generate different mishybridization formations during the assembly.

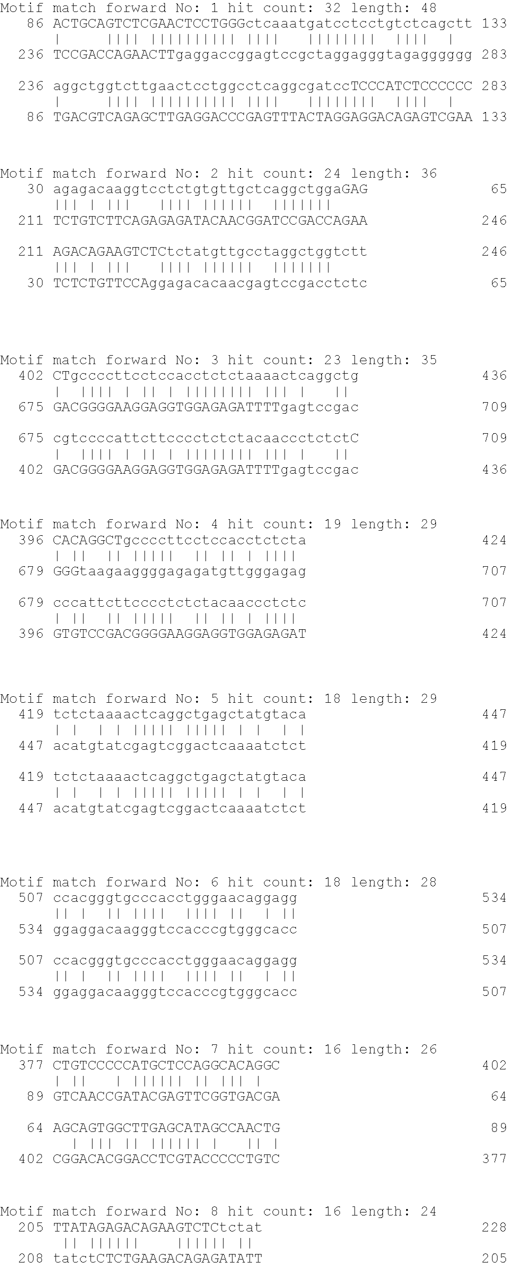

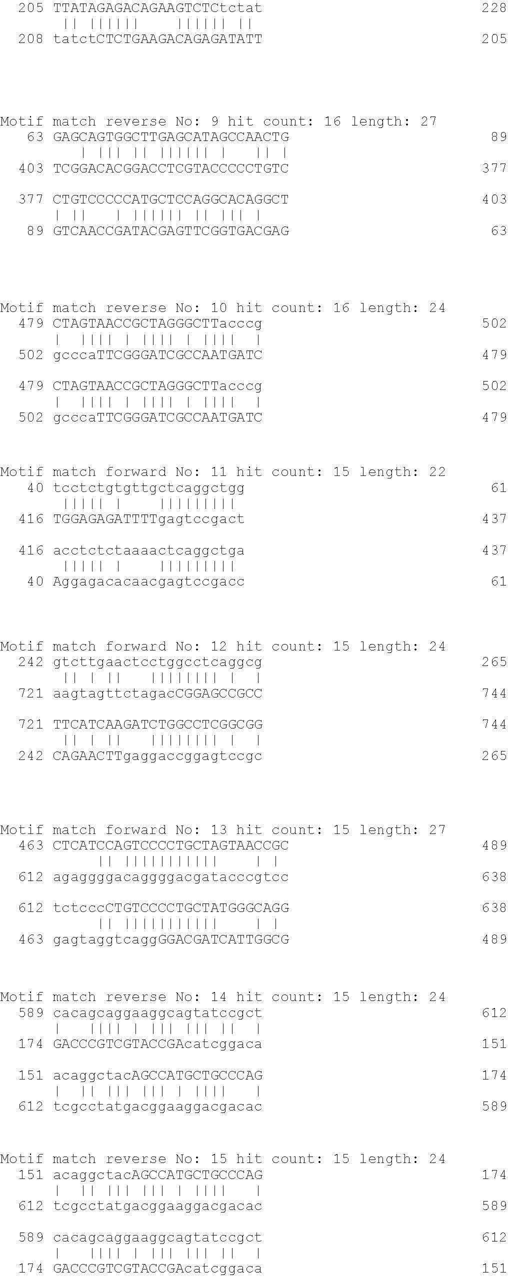

TABLE S5
Partial list of potential mishybridizations for PKB2 gene synthesis predicted by TmPrime
gene synthesis software (http://prime.ibn.a-star.edu.sg).

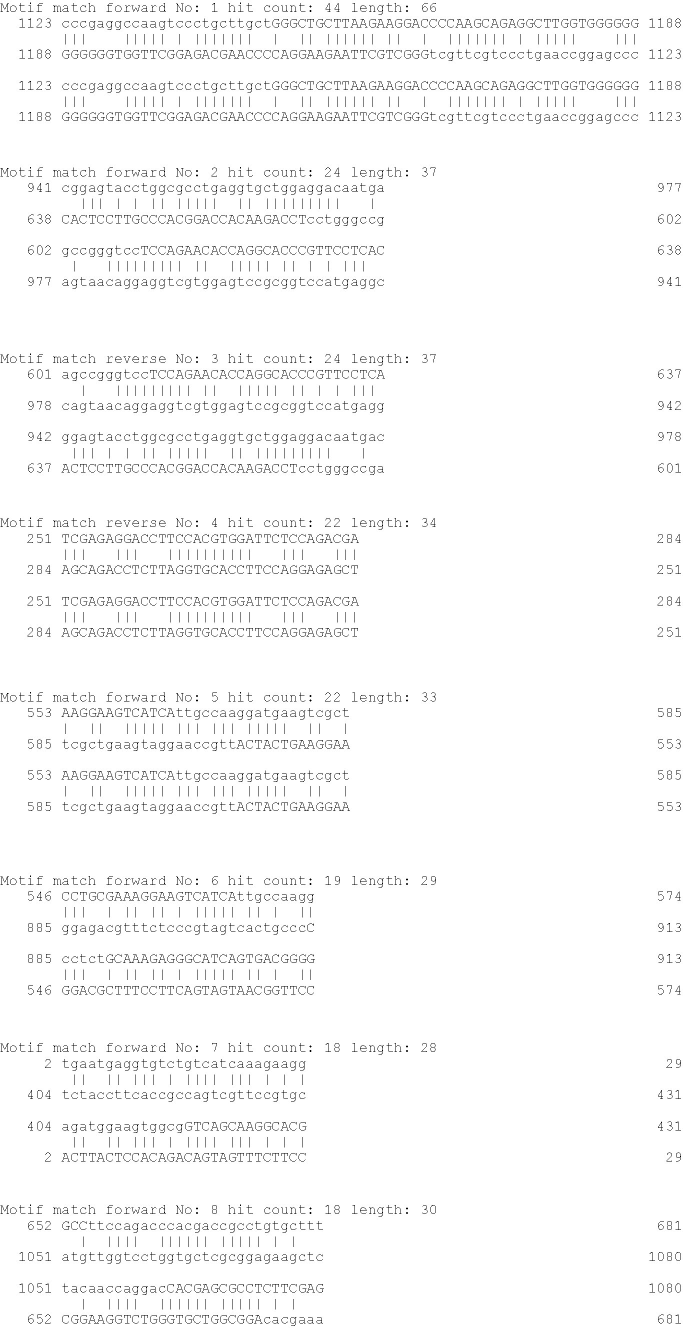

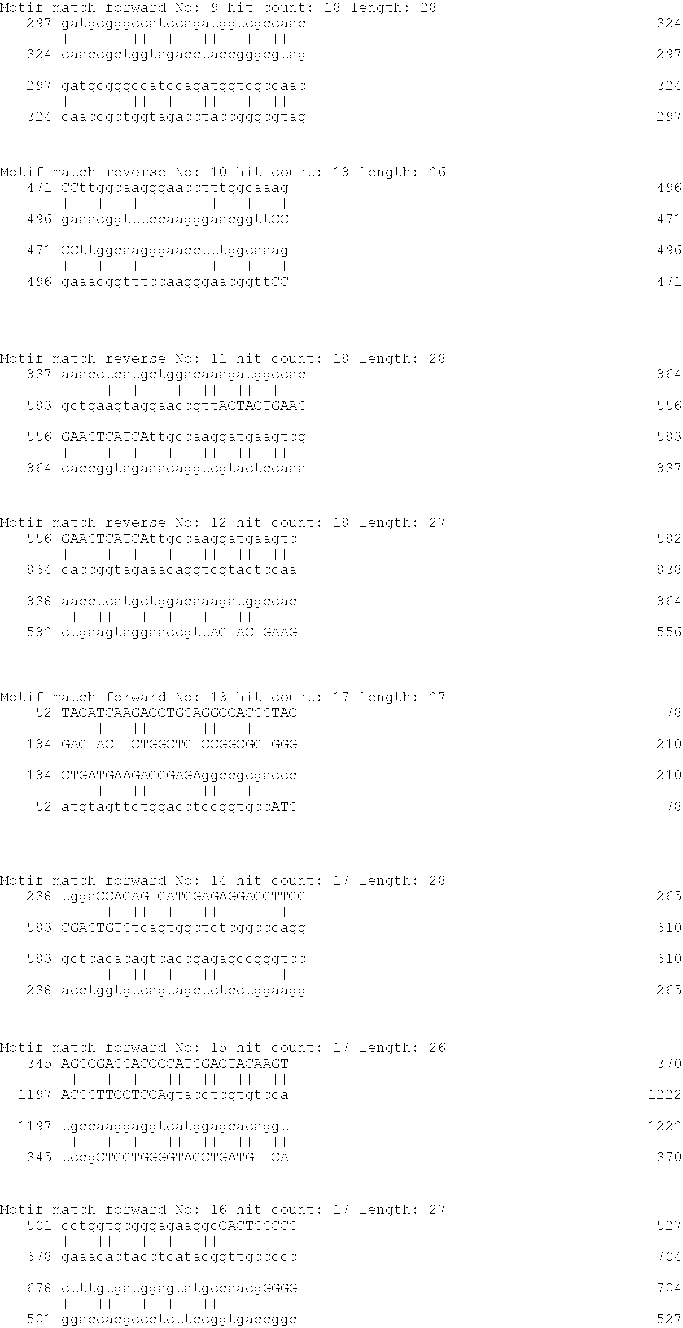

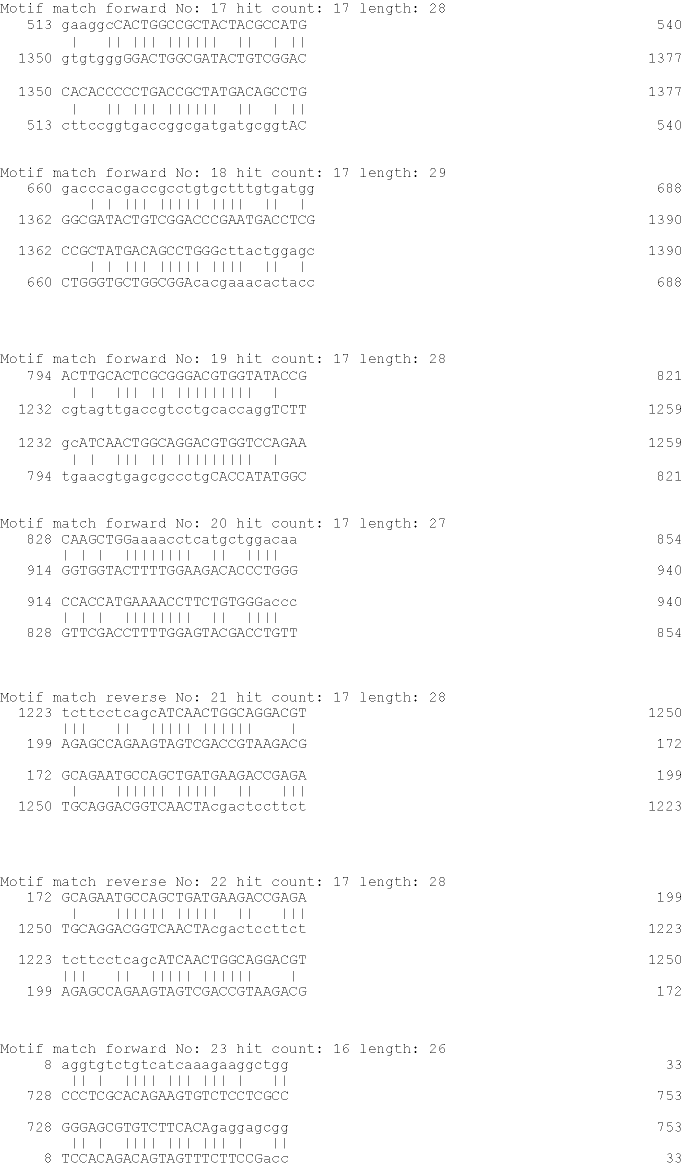

TABLE S6
Summary of melting temperatures of S100A4-1,
S100A4-2 and PKB2 oligonucleotide sets at oligonucleotide
concentrations of 10 nM and 1 nM.

| | | Average | Std. | | Minimum | Maximum |
|---|---|---|---|---|---|---|
| | [Oligos] | Tₘ | of Tₘ | ΔTₘ | Tₘ | Tₘ |
| Gene | (nM) | (° C.) | (° C.) | (° C.) | (° C.) | (° C.) |
| S100A4-1 | 10 | 66.81 | 3.0 | 9.1 | 61.64 | 70.73 |
| | 1 | 64.04 | 3.05 | 9.93 | 58.56 | 68.5 |
| S100A4-2 | 10 | 65.25 | 0.48 | 2.03 | 64.52 | 66.55 |
| | 1 | 62.31 | 0.55 | 2.60 | 60.96 | 63.57 |
| PKB2 | 10 | 66.31 | 0.56 | 1.91 | 65.37 | 67.28 |
| | 1 | 63.37 | 0.70 | 2.86 | 61.85 | 64.71 |

1. A method of synthesising a nucleic acid molecule by a polymerase chain reaction (PCR), comprising:
(a) assembling a nucleic acid template by PCR comprising subjecting a PCR reaction mixture comprising a set of assembly oligonucleotides and a set of amplification primers in the presence of a nucleic acid polymerase to reaction conditions that allow hybridization of the assembly oligonucleotides to each other (annealing) and nucleic acid polymerization;
wherein the set of assembly oligonucleotides comprises at least two distinct outer assembly oligonucleotides and a multitude of distinct inner assembly oligonucleotides;
wherein each of the inner assembly oligonucleotides comprises on its 5′ end a first nucleic acid sequence complementary to a nucleic acid sequence on the 5′ end of another first inner assembly oligonucleotide and, on its 3′ end, a second nucleic acid sequence complementary to a nucleic acid sequence on the 3′ end of another second inner or one of the at least two outer assembly oligonucleotide to allow hybridization to each other under hybridization conditions;
wherein each of the outer assembly oligonucleotides comprises on its 3′ end a nucleic acid sequence complementary to a nucleic acid sequence on the 3′ end of an inner assembly oligonucleotide to allow hybridization under hybridization conditions; and
wherein each of the amplification primers comprises on its 3′ end a nucleic acid sequence that is identical to a sequence on the 5′ end of an outer assembly oligonucleotide and a nucleic acid sequence that is not identical to a nucleic acid sequence of any one of the assembly oligonucleotides and not complementary to a nucleic acid sequence of any one of the assembly oligonucleotides, wherein each melting temperature of the nucleic acid sequences of the amplification primers identical to part of the sequence of an outer assembly oligonucleotide is lower than each melting temperature of the complementary sequences of the assembly oligonucleotides, and wherein each of the melting temperatures of the complete amplification primer sequences is higher than or equal to the average melting temperatures of the complementary regions of the assembly oligonucleotides or higher than or equal to the lowest melting temperature of the complementary regions of the assembly oligonucleotides; and
(b) amplifying the assembled nucleic acid template by PCR;
wherein the reaction conditions in (a) and (b) are the same; and
wherein the reaction conditions in (a) and (b) include an annealing temperature higher than each melting temperature of the nucleic acid sequences of the amplification primers that are identical to part of the sequence of an outer assembly oligonucleotide but lower than or equal to each melting temperature of the nucleic acid sequences of the complete amplification primers.

2. The method of claim 1, wherein the assembly oligonucleotides are each about 30 to about 100 nucleotides, about 35 to about 95, about 40 to about 90, about 45 to about 85, about 50 to about 80, about 55 to about 75, about 50 to about 70, or about 55 to about 65 nucleotides in length.

3. The method of claim 1, wherein the complementary regions of the assembly oligonucleotides are each about 10 to about 50, about 15 to about 45, about 20 to about 40, about 25 to about 35, or about 20 to about 30 nucleotides in length.

4. The method of claim 1, wherein the nucleic acid sequence of the amplification primers that is not identical to a nucleic acid sequence of any one of the assembly oligonucleotides and not complementary to a nucleic acid sequence of any one of the assembly oligonucleotides is at least 5 nucleotides in length.

5. The method of claim 1, wherein the synthesized nucleic acid molecule is a double-stranded nucleic acid molecule.

6. The method of claim 5, wherein the synthesized nucleic acid molecule is a double-stranded DNA molecule.

7. The method of claim 1, wherein the annealing temperature employed in (b) is not lower than that employed in (a).

8. The method of claim 1, wherein the difference between the melting temperatures of the distinct assembly oligonucleotides is lower than or equal to about 10° C.

9. The method of claim 8, wherein the difference between the melting temperatures of the distinct assembly oligonucleotides is in the range of about 5° C. to about 3° C.

10. The method of claim 1, wherein the average melting temperature of the complementary region(s) of the assembly oligonucleotides is in the range of about 65° C. to about 80° C.

11. The method of claim 1, wherein the difference in the melting temperature of each of the complementary region(s) of the assembly oligonucleotides and the first melting temperature of each of the amplification primers is at least about 5° C.

12. The method of claim 1, wherein the difference in the melting temperature of each of the complementary region(s) of the assembly oligonucleotides and the first melting temperature of each of the amplification primers is from about 5° C. to about 20° C.

13. The method of claim 1, wherein the melting temperature of each of the full length amplification primers is equal to or higher than the average melting temperature of the complementary region(s) of the assembly oligonucleotides or equal to or higher than the lowest melting temperature of the complementary region(s) of the assembly oligonucleotides and is in the range of about 65° C. to about 80° C.

14. The method of claim 1, wherein the annealing temperature is at least about 5° C. higher than the average first melting temperature of the amplification primer set or each individual first melting temperature of the amplification primers.

15. The method of claim 1, wherein the annealing temperature is equal to or lower than the average melting temperature of the complementary region(s) of the assembly oligonucleotides.

16. The method of claim 1, wherein the annealing temperature is slightly higher than the average melting temperature of the complementary region(s) of the assembly oligonucleotides.

17. The method of claim 1, wherein the annealing temperature is about 72° C.

18. The method of claim 1, wherein the concentration of the set of assembly oligonucleotides in the PCR mixture is from about 0.05 nM to about 100 nM.

19. The method of claim 1, wherein the concentration of the set of amplification primers in the PCR mixture is from about 100 nM to about 1 μM.

20. The method of claim 1, wherein said method comprises conducting from about 15 to about 50 PCR cycles.

21. The method of claim 1, wherein the nucleic acid molecule to be synthesized is about 500 to about 2000 nucleotides long.

22. The method of claim 1, wherein the PCR is hot-start PCR.

23. The method of claim 1, wherein the PCR is real time PCR (RT-PCR).

24. The method of claim 23, wherein the method comprises the use of a fluorescent DNA marker.

25. The method of claim 24, wherein the marker is LCGreen I.

26. The method of claim 1, wherein the nucleic acid molecule to be synthesized is about 500 to about 4000 nucleotides in length.

27. A kit comprising a set of assembly oligonucleotides and a set of amplification primers,
wherein the set of assembly oligonucleotides comprises at least two distinct outer assembly oligonucleotides and a multitude of distinct inner assembly oligonucleotides;
wherein each of the inner assembly oligonucleotides comprises on its 5′ end a first nucleic acid sequence complementary to a nucleic acid sequence on the 5′ end of another first inner assembly oligonucleotide and, on its 3′ end, a second nucleic acid sequence complementary to a nucleic acid sequence on the 3′ end of another second inner or one of the at least two outer assembly oligonucleotides to allow hybridization to each other under hybridization conditions;
wherein each of the outer assembly oligonucleotides comprises on its 3′ end a nucleic acid sequence complementary to a nucleic acid sequence on the 3′ end of an inner assembly oligonucleotide to allow hybridization under hybridization conditions; and
wherein each of the amplification primers comprises on its 3′ end a nucleic acid sequence that is identical to a sequence on the 5′ end of an outer assembly oligonucleotide and a nucleic acid sequence that is not identical to a nucleic acid sequence of any one of the assembly oligonucleotides and not complementary to a nucleic acid sequence of any one of the assembly oligonucleotides; wherein each melting temperature of the nucleic acid sequences of the amplification primers identical to the 5′ end of an outer assembly oligonucleotide is lower than each melting temperature of the complementary sequences of the assembly oligonucleotides; and wherein each of the melting temperatures of the complete amplification primer sequences is higher than or equal to the lowest melting temperature of the complementary sequences of the assembly oligonucleotides.


SEQUENCE LISTING (133 sequences)

SEQ ID NO: 1 - DNA, 38 bp, Artificial
  S100A4 primer 1-step 1
gtttttctttctgaatctttatttttttaagagacaag

SEQ ID NO: 2 - DNA, 14 bp, Artificial
  S100A4 primer 1-step 2
aagcttggccgccg

SEQ ID NO: 3 - DNA, 61 bp, Artificial
  S100A4 primer ATD 1
agtagtagtagtagtagtagtagtagtagtagtgtttttgtttctgaatctttattttttt

SEQ ID NO: 4 - DNA, 44 bp, Artificial
  S100A4 primer ATD 2
agaagaagaagaagaagaagaagaagaagaaagcttggccgccg

SEQ ID NO: 5 - DNA, 30 bp, Artificial
  PKB2 primer 1-step 1
atgaatgaggtgtctgtcatcaaagaaggc

SEQ ID NO: 6 - DNA, 19 bp, Artificial
  PKB2 primer 1-step 2
tcactcgcggatgctggcc

SEQ ID NO: 7 - DNA, 53 bp, Artificial
  PKB2 primer ATD 1
agaagaagaagaagaagaagaagaagaagaagaatgaatgaggtgtctgtcat

SEQ ID NO: 8 - DNA, 52 bp, Artificial
  PKB2 primer ATD 2
agtagtagtagtagtagtagtagtagtagtagtagttcactcgcggatgctg

SEQ ID NO: 9 - DNA, 59 bp, Artificial
  S100A4-1 assembly F1
gtttttgtttctgaatctttatttttttaagagacaaggtcctctgtgttgctcaggct

SEQ ID NO: 10 - DNA, 41 bp, Artificial
  S100A4-1 assembly R1
tgctcaagccactgctctccagcctgagcaacacagaggac

SEQ ID NO: 11 - DNA, 42 bp, Artificial
  S100A4-1 assembly F2
ggagagcagtggcttgagcatagccaactgcagtctcgaact

SEQ ID NO: 12 - DNA, 44 bp, Artificial
  S100A4-1 assembly R2
aggaggatcatttgagcccaggagttcgagactgcagttggcta

SEQ ID NO: 13 - DNA, 45 bp, Artificial
  S100A4-1 assembly F3
cctgggctcaaatgatcctcctgtctcagcttcctgactagctgg

SEQ ID NO: 14 - DNA, 44 bp, Artificial
  S100A4-1 assembly R3
gcatggctgtagcctgtagtcccagctagtcaggaagctgagac

SEQ ID NO: 15 - DNA, 54 bp, Artificial
  S100A4-1 assembly F4
gactacaggctacagccatgctgcccagctaattaaaaaaaaaaattgtttttc

SEQ ID NO: 16 - DNA, 66 bp, Artificial
  S100A4-1 assembly R4
gcaacatagagagacttctgtctctataaaaaggaaaaacaatttttttttttaattagctgggca

SEQ ID NO: 17 - DNA, 56 bp, Artificial
  S100A4-1 assembly F5
ctttttatagagacagaagtctctctatgttgcctaggctggtcttgaactcctgg

SEQ ID NO: 18 - DNA, 45 bp, Artificial
  S100A4-1 assembly R5
gagatgggaggatcgcctgaggccaggagttcaagaccagcctag

SEQ ID NO: 19 - DNA, 49 bp, Artificial
  S100A4-1 assembly F6
cctcaggcgatcctcccatctcccccctagcttttgtgtcaccacattt

SEQ ID NO: 20 - DNA, 50 bp, Artificial
  S100A4-1 assembly R6
tgacaggtgggagattgccctggaaatgtggtgacacaaaagctaggggg

SEQ ID NO: 21 - DNA, 44 bp, Artificial
  S100A4-1 assembly F7
ccagggcaatctcccacctgtcacccaccaccccctgcatctcc

SEQ ID NO: 22 - DNA, 48 bp, Artificial
  S100A4-1 assembly R7
ggagtagtcccatggggacctaggaaaggagatgcagggggtggtggg

SEQ ID NO: 23 - DNA, 49 bp, Artificial
  S100A4-1 assembly F8
tttcctaggtccccatgggactactccctgtcccccatgctccaggcac

SEQ ID NO: 24 - DNA, 43 bp, Artificial
  S100A4-1 assembly R8
aggtggaggaaggggcagcctgtgcctggagcatgggggacag

SEQ ID NO: 25 - DNA, 54 bp, Artificial
  S100A4-1 assembly F9
aggctgccccttcctccacctctctaaaactcaggctgagctatgtacactggg

SEQ ID NO: 26 - DNA, 57 bp, Artificial
  S100A4-1 assembly R9
ggggactggatgagatgggcaccacccagtgtacatagctcagcctgagttttagag

SEQ ID NO: 27 - DNA, 54 bp, Artificial
  S100A4-1 assembly F10
tggtgcccatctcatccagtcccctgctagtaaccgctagggcttacccgttac

SEQ ID NO: 28 - DNA, 51 bp, Artificial
  S100A4-1 assembly R10
ttcccaggtgggcacccgtgggtaacgggtaagccctagcggttactagca

SEQ ID NO: 29 - DNA, 45 bp, Artificial
  S100A4-1 assembly F11
ccacgggtgcccacctgggaacaggaggcttggttccacggctgg

SEQ ID NO: 30 - DNA, 45 bp, Artificial
  S100A4-1 assembly R11
gccacagcaccctccaccagcccagccgtggaaccaagcctcctg

SEQ ID NO: 31 - DNA, 45 bp, Artificial
  S100A4-1 assembly F12
gctggtggagggtgctgtggcacttaccgcatcagcccacagcag

SEQ ID NO: 32 - DNA, 50 bp, Artificial
  S100A4-1 assembly R12
gacaggggagagcggatactgccttcctgctgtgggctgatgcggtaagt

SEQ ID NO: 33 - DNA, 47 bp, Artificial
  S100A4-1 assembly F13
gaaggcagtatccgctctcccctgtcccctgctatgggcagggcctg

SEQ ID NO: 34 - DNA, 52 bp, Artificial
  S100A4-1 assembly R13
gcccagaggtctgacctatttataccccagccaggccctgcccatagcaggg

SEQ ID NO: 35 - DNA, 59 bp, Artificial
  S100A4-1 assembly F14
gctggggtataaataggtcagacctctgggccgtccccattcttcccctctctacaacc

SEQ ID NO: 36 - DNA, 59 bp, Artificial
  S100A4-1 assembly R14
agatcttgatgaagaagcgctgaggagagagggttgtagagaggggaagaatggggacg

SEQ ID NO: 37 - DNA, 50 bp, Artificial
  S100A4-1 assembly F15
ctctctcctcagcgcttcttcatcaagatctggcctcggcggccaagctt

SEQ ID NO: 38 - DNA, 19 bp, Artificial
  S100A4-1 assembly R15
aagcttggccgccgaggcc

SEQ ID NO: 39 - DNA, 19 bp, Artificial
  S100A4-1 primer 1-step R
aagcttggccgccgaggcc

SEQ ID NO: 40 - DNA, 62 bp, Artificial
  S100A4-2 assembly F1
gtttttgtttctgaatctttatttttttaagagacaaggtcctctgtgttgctcaggctgga

SEQ ID NO: 41 - DNA, 44 bp, Artificial
  S100A4-2 assembly R1
ggctatgctcaagccactgctctccagcctgagcaacacagagg

SEQ ID NO: 42 - DNA, 45 bp, Artificial
  S100A4-2 assembly F2
gagcagtggcttgagcatagccaactgcagtctcgaactcctggg

SEQ ID NO: 43 - DNA, 50 bp, Artificial
  S100A4-2 assembly R2
gaagctgagacaggaggatcatttgagcccaggagttcgagactgcagtt

SEQ ID NO: 44 - DNA, 52 bp, Artificial
  S100A4-2 assembly F3
ctcaaatgatcctcctgtctcagcttcctgactagctgggactacaggctac

SEQ ID NO: 45 - DNA, 50 bp, Artificial
  S100A4-2 assembly R3
ttttaattagctgggcagcatggctgtagcctgtagtcccagctagtcag

SEQ ID NO: 46 - DNA, 64 bp, Artificial
  S100A4-2 assembly F4
agccatgctgcccagctaattaaaaaaaaaaattgtttttcctttttatagagacagaagtctc

SEQ ID NO: 47 - DNA, 65 bp, Artificial
  S100A4-2 assembly R4
ttcaagaccagcctaggcaacatagagagacttctgtctctataaaaaggaaaaacaattttttt

SEQ ID NO: 48 - DNA, 46 bp, Artificial
  S100A4-2 assembly F5
tctatgttgcctaggctggtcttgaactcctggcctcaggcgatcc

SEQ ID NO: 49 - DNA, 43 bp, Artificial
  S100A4-2 assembly R5
caaaagctaggggggagatgggaggatcgcctgaggccaggag

SEQ ID NO: 50 - DNA, 48 bp, Artificial
  S100A4-2 assembly F6
tcccatctcccccctagcttttgtgtcaccacatttccagggcaatct

SEQ ID NO: 51 - DNA, 45 bp, Artificial
  S100A4-2 assembly R6
ggtggtgggtgacaggtgggagattgccctggaaatgtggtgaca

SEQ ID NO: 52 - DNA, 44 bp, Artificial
  S100A4-2 assembly F7
cccacctgtcacccaccaccccctgcatctcctttcctaggtcc

SEQ ID NO: 53 - DNA, 46 bp, Artificial
  S100A4-2 assembly R7
gggacagggagtagtcccatggggacctaggaaaggagatgcaggg

SEQ ID NO: 54 - DNA, 42 bp, Artificial
  S100A4-2 assembly F8
ccatgggactactccctgtcccccatgctccaggcacaggct

SEQ ID NO: 55 - DNA, 44 bp, Artificial
  S100A4-2 assembly R8
ttttagagaggtggaggaaggggcagcctgtgcctggagcatgg

SEQ ID NO: 56 - DNA, 49 bp, Artificial
  S100A4-2 assembly F9
gccccttcctccacctctctaaaactcaggctgagctatgtacactggg

SEQ ID NO: 57 - DNA, 47 bp, Artificial
  S100A4-2 assembly R9
ggactggatgagatgggcaccacccagtgtacatagctcagcctgag

SEQ ID NO: 58 - DNA, 45 bp, Artificial
  S100A4-2 assembly F10
tggtgcccatctcatccagtcccctgctagtaaccgctagggctt

SEQ ID NO: 59 - DNA, 42 bp, Artificial
  S100A4-2 assembly R10
gcacccgtgggtaacgggtaagccctagcggttactagcagg

SEQ ID NO: 60 - DNA, 40 bp, Artificial
  S100A4-2 assembly F11
acccgttacccacgggtgcccacctgggaacaggaggctt

SEQ ID NO: 61 - DNA, 40 bp, Artificial
  S100A4-2 assembly R11
ccagcccagccgtggaaccaagcctcctgttcccaggtgg

SEQ ID NO: 62 - DNA, 39 bp, Artificial
  S100A4-2 assembly F12
ggttccacggctgggctggtggagggtgctgtggcactt

SEQ ID NO: 63 - DNA, 39 bp, Artificial
  S100A4-2 assembly R12
tgctgtgggctgatgcggtaagtgccacagcaccctcca

SEQ ID NO: 64 - DNA, 41 bp, Artificial
  S100A4-2 assembly F13
accgcatcagcccacagcaggaaggcagtatccgctctccc

SEQ ID NO: 65 - DNA, 43 bp, Artificial
  S100A4-2 assembly R13
cctgcccatagcaggggacaggggagagcggatactgccttcc

SEQ ID NO: 66 - DNA, 46 bp, Artificial
  S100A4-2 assembly F14
ctgtcccctgctatgggcagggcctggctggggtataaataggtca

SEQ ID NO: 67 - DNA, 43 bp, Artificial
  S100A4-2 assembly R14
ggggacggcccagaggtctgacctatttataccccagccaggc

SEQ ID NO: 68 - DNA, 45 bp, Artificial
  S100A4-2 assembly F15
gacctctgggccgtccccattcttcccctctctacaaccctctct

SEQ ID NO: 69 - DNA, 53 bp, Artificial
  S100A4-2 assembly R15
cagatcttgatgaagaagcgctgaggagagagggttgtagagaggggaagaat

SEQ ID NO: 70 - DNA, 44 bp, Artificial
  S100A4-2 assembly F16
cctcagcgcttcttcatcaagatctggcctcggcggccaagctt

SEQ ID NO: 71 - DNA, 18 bp, Artificial
  S100A4-2 assembly R16
aagcttggccgccgaggc

SEQ ID NO: 72 - DNA, 51 bp, Artificial
  PKB2 assembly F1
atgaatgaggtgtctgtcatcaaagaaggctggctccacaagcgtggtgaa

SEQ ID NO: 73 - DNA, 45 bp, Artificial
  PKB2 assembly R1
ccgtggcctccaggtcttgatgtattcaccacgcttgtggagcca

SEQ ID NO: 74 - DNA, 47 bp, Artificial
  PKB2 assembly F2
tacatcaagacctggaggccacggtacttcctgctgaagagcgacgg

SEQ ID NO: 75 - DNA, 49 bp, Artificial
  PKB2 assembly R2
gcctctccttgtacccaatgaaggagccgtcgctcttcagcaggaagta

SEQ ID NO: 76 - DNA, 49 bp, Artificial
  PKB2 assembly F3
ctccttcattgggtacaaggagaggcccgaggcccctgatcagactcta

SEQ ID NO: 77 - DNA, 49 bp, Artificial
  PKB2 assembly R3
gctacggagaagttgtttaaggggggtagagtctgatcaggggcctcgg

SEQ ID NO: 78 - DNA, 52 bp, Artificial
  PKB2 assembly F4
ccccccttaaacaacttctccgtagcagaatgccagctgatgaagaccgaga

SEQ ID NO: 79 - DNA, 46 bp, Artificial
  PKB2 assembly R4
aaaggtgttgggtcgcggcctctcggtcttcatcagctggcattct

SEQ ID NO: 80 - DNA, 42 bp, Artificial
  PKB2 assembly F5
ggccgcgacccaacacctttgtcatacgctgcctgcagtgga

SEQ ID NO: 81 - DNA, 47 bp, Artificial
  PKB2 assembly R5
tggaaggtcctctcgatgactgtggtccactgcaggcagcgtatgac

SEQ ID NO: 82 - DNA, 49 bp, Artificial
  PKB2 assembly F6
ccacagtcatcgagaggaccttccacgtggattctccagacgagaggga

SEQ ID NO: 83 - DNA, 44 bp, Artificial
  PKB2 assembly R6
ggatggcccgcatccactcctccctctcgtctggagaatccacg

SEQ ID NO: 84 - DNA, 42 bp, Artificial
  PKB2 assembly F7
ggagtggatgcgggccatccagatggtcgccaacagcctcaa

SEQ ID NO: 85 - DNA, 38 bp, Artificial
  PKB2 assembly R7
gcctggggcccgctgcttgaggctgttggcgaccatct

SEQ ID NO: 86 - DNA, 41 bp, Artificial
  PKB2 assembly F8
gcagcgggccccaggcgaggaccccatggactacaagtgtg

SEQ ID NO: 87 - DNA, 46 bp, Artificial
  PKB2 assembly R8
tggaggagtcactgggggagccacacttgtagtccatggggtcctc

SEQ ID NO: 88 - DNA, 44 bp, Artificial
  PKB2 assembly F9
gctcccccagtgactcctccacgactgaggagatggaagtggcg

SEQ ID NO: 89 - DNA, 46 bp, Artificial
  PKB2 assembly R9
actttagcccgtgccttgctgaccgccacttccatctcctcagtcg

SEQ ID NO: 90 - DNA, 55 bp, Artificial
  PKB2 assembly F10
gtcagcaaggcacgggctaaagtgaccatgaatgacttcgactatctcaaactcc

SEQ ID NO: 91 - DNA, 57 bp, Artificial
  PKB2 assembly R10
actttgccaaaggttcccttgccaaggagtttgagatagtcgaagtcattcatggtc

SEQ ID NO: 92 - DNA, 46 bp, Artificial
  PKB2 assembly F11
ttggcaagggaacctttggcaaagtcatcctggtgcgggagaaggc

SEQ ID NO: 93 - DNA, 41 bp, Artificial
  PKB2 assembly R11
tggcgtagtagcggccagtggccttctcccgcaccaggatg

SEQ ID NO: 94 - DNA, 47 bp, Artificial
  PKB2 assembly F12
cactggccgctactacgccatgaagatcctgcgaaaggaagtcatca

SEQ ID NO: 95 - DNA, 52 bp, Artificial
  PKB2 assembly R12
gtgtgagcgacttcatccttggcaatgatgacttcctttcgcaggatcttca

SEQ ID NO: 96 - DNA, 45 bp, Artificial
  PKB2 assembly F13
ttgccaaggatgaagtcgctcacacagtcaccgagagccgggtcc

SEQ ID NO: 97 - DNA, 41 bp, Artificial
  PKB2 assembly R13
acgggtgcctggtgttctggaggacccggctctcggtgact

SEQ ID NO: 98 - DNA, 44 bp, Artificial
  PKB2 assembly F14
tccagaacaccaggcacccgttcctcactgcgctgaagtatgcc

SEQ ID NO: 99 - DNA, 43 bp, Artificial
  PKB2 assembly R14
aggcggtcgtgggtctggaaggcatacttcagcgcagtgagga

SEQ ID NO: 100 - DNA, 46 bp, Artificial
  PKB2 assembly F15
ttccagacccacgaccgcctgtgctttgtgatggagtatgccaacg

SEQ ID NO: 101 - DNA, 49 bp, Artificial
  PKB2 assembly R15
caggtggaagaacagctcacccccgttggcatactccatcacaaagcac

SEQ ID NO: 102 - DNA, 44 bp, Artificial
  PKB2 assembly F16
ggggtgagctgttcttccacctgtcccgggagcgtgtcttcaca

SEQ ID NO: 103 - DNA, 40 bp, Artificial
  PKB2 assembly R16
aaaaccgggcccgctcctctgtgaagacacgctcccggga

SEQ ID NO: 104 - DNA, 43 bp, Artificial
  PKB2 assembly F17
gaggagcgggcccggttttatggtgcagagattgtctcggctc

SEQ ID NO: 105 - DNA, 47 bp, Artificial
  PKB2 assembly R17
gtcccgcgagtgcaagtactcaagagccgagacaatctctgcaccat

SEQ ID NO: 106 - DNA, 48 bp, Artificial
  PKB2 assembly F18
ttgagtacttgcactcgcgggacgtggtataccgcgacatcaagctgg

SEQ ID NO: 107 - DNA, 51 bp, Artificial
  PKB2 assembly R18
gccatctttgtccagcatgaggttttccagcttgatgtcgcggtataccac

SEQ ID NO: 108 - DNA, 54 bp, Artificial
  PKB2 assembly F19
aaaacctcatgctggacaaagatggccacatcaagatcactgactttggcctct

SEQ ID NO: 109 - DNA, 51 bp, Artificial
  PKB2 assembly R19
cccgtcactgatgccctctttgcagaggccaaagtcagtgatcttgatgtg

SEQ ID NO: 110 - DNA, 47 bp, Artificial
  PKB2 assembly F20
gcaaagagggcatcagtgacggggccaccatgaaaaccttctgtggg

SEQ ID NO: 111 - DNA, 43 bp, Artificial
  PKB2 assembly R20
gcgccaggtactccggggtcccacagaaggttttcatggtggc

SEQ ID NO: 112 - DNA, 43 bp, Artificial
  PKB2 assembly F21
accccggagtacctggcgcctgaggtgctggaggacaatgact

SEQ ID NO: 113 - DNA, 42 bp, Artificial
  PKB2 assembly R21
agtccacggcccggccatagtcattgtcctccagcacctcag

SEQ ID NO: 114 - DNA, 36 bp, Artificial
  PKB2 assembly F22
atggccgggccgtggactggtgggggctgggtgtgg

SEQ ID NO: 115 - DNA, 42 bp, Artificial
  PKB2 assembly R22
ggccgcacatcatctcgtacatgaccacacccagcccccacc

SEQ ID NO: 116 - DNA, 47 bp, Artificial
  PKB2 assembly F23
tcatgtacgagatgatgtgcggccgcctgcccttctacaaccaggac

SEQ ID NO: 117 - DNA, 43 bp, Artificial
  PKB2 assembly R23
agctcgaagaggcgctcgtggtcctggttgtagaagggcaggc

SEQ ID NO: 118 - DNA, 46 bp, Artificial
  PKB2 assembly F24
cacgagcgcctcttcgagctcatcctcatggaagagatccgcttcc

SEQ ID NO: 119 - DNA, 43 bp, Artificial
  PKB2 assembly R24
ggggctgagcgtgcgcgggaagcggatctcttccatgaggatg

SEQ ID NO: 120 - DNA, 38 bp, Artificial
  PKB2 assembly F25
cgcgcacgctcagccccgaggccaagtccctgcttgct

SEQ ID NO: 121 - DNA, 44 bp, Artificial
  PKB2 assembly R25
ttggggtccttcttaagcagcccagcaagcagggacttggcctc

SEQ ID NO: 122 - DNA, 42 bp, Artificial
  PKB2 assembly F26
gggctgcttaagaaggaccccaagcagaggcttggtgggggg

SEQ ID NO: 123 - DNA, 39 bp, Artificial
  PKB2 assembly R26
acctccttggcatcgctgggccccccaccaagcctctgc

SEQ ID NO: 124 - DNA, 45 bp, Artificial
  PKB2 assembly F27
cccagcgatgccaaggaggtcatggagcacaggttcttcctcagc

SEQ ID NO: 125 - DNA, 47 bp, Artificial
  PKB2 assembly R27
ggaccacgtcctgccagttgatgctgaggaagaacctgtgctccatg

SEQ ID NO: 126 - DNA, 46 bp, Artificial
  PKB2 assembly F28
atcaactggcaggacgtggtccagaagaagctcctgccacccttca

SEQ ID NO: 127 - DNA, 47 bp, Artificial
  PKB2 assembly R28
gacctcggacgtgacctgaggtttgaagggtggcaggagcttcttct

SEQ ID NO: 128 - DNA, 54 bp, Artificial
  PKB2 assembly F29
aacctcaggtcacgtccgaggtcgacacaaggtacttcgatgatgaatttaccg

SEQ ID NO: 129 - DNA, 55 bp, Artificial
  PKB2 assembly R29
ggggtgtgattgtgatggactgggcggtaaattcatcatcgaagtaccttgtgtc

SEQ ID NO: 130 - DNA, 46 bp, Artificial
  PKB2 assembly F30
cccagtccatcacaatcacaccccctgaccgctatgacagcctggg

SEQ ID NO: 131 - DNA, 45 bp, Artificial
  PKB2 assembly R30
tccgctggtccagctccagtaagcccaggctgtcatagcggtcag

SEQ ID NO: 132 - DNA, 48 bp, Artificial
  PKB2 assembly F31
cttactggagctggaccagcggacccacttcccccagttctcctactc

SEQ ID NO: 133 - DNA, 44 bp, Artificial
  PKB2 assembly R31
tcactcgcggatgctggccgagtaggagaactgggggaagtggg